(19) United States
(12) Patent Application Publication
(10) Pub. No.: US 2011/0245335 A1
(43) Pub. Date: Oct. 6, 2011

(54) NEW INHIBITORS FOR TREATING DISEASES ASSOCIATED WITH AN EXCESS TRANSPORT OF HYALURONAN

(76) Inventor: Peter Prehm, Nottuln (DE)

(21) Appl. No.: 13/139,164

(22) PCT Filed: Dec. 14, 2009
(86) PCT No.: PCT/EP2009/067113

§ 371 Date: Jun. 10, 2011

(30) Foreign Application Priority Data
Dec. 12, 2008 (EP) 08171524.5

(51) Int. Cl. A61K 31/351; C07D 315/00; C07C 63/33; A61K 31/195; C07C 213/00; C07C 233/00; A61K 31/165; A61K 31/135; A61P 35/04; A61P 29/00; A61P 19/02

(52) U.S. Cl. 514/459; 549/417; 562/405; 514/563; 564/430; 564/161; 514/619; 514/646

(57) ABSTRACT

The present invention relates in general to a compound which is characterized by a formula selected from the following formulas A, B, C, D, E or F; or a pharmaceutically acceptable salt thereof. The present invention further relates to pharmaceutical composition comprising the inhibitor(s) of the invention and to their use in the treatment of (for treating) a disease which is associated with an excess transport of hyaluronan across a lipid bilayer. The present invention furthermore relates to the use of at least one inhibitor of the invention for the preparation of a pharmaceutical composition for the treatment of (for treating) a disease which is associated with an excess transport of hyaluronan across a lipid bilayer. The present invention also relates to a method for manufacturing a pharmaceutical composition comprising the steps of formulating the inhibitor defined herein in a pharmaceutically acceptable form.

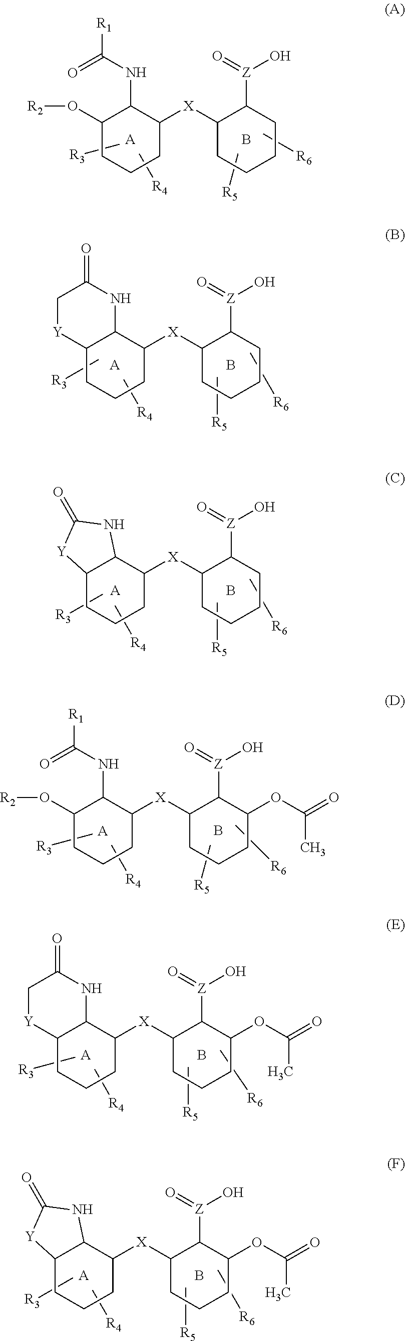

The present invention relates in general to a compound which is characterized by a formula selected from the following formulas A, B, C, D, E or F:

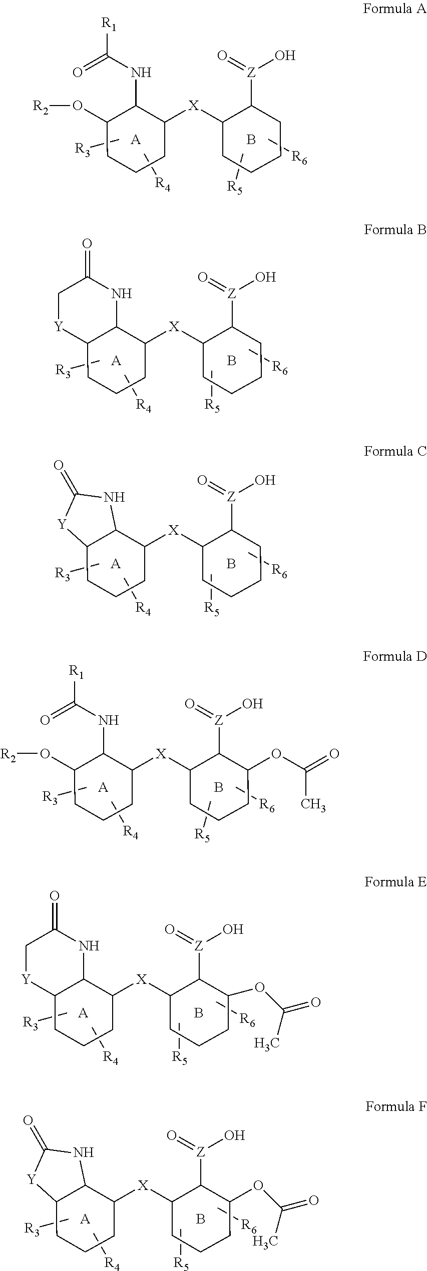

or a pharmaceutically acceptable salt thereof. The present invention further relates to pharmaceutical composition comprising the inhibitor(s) of the invention and to their use in the treatment of (for treating) a disease which is associated with an excess transport of hyaluronan across a lipid bilayer. The present invention furthermore relates to the use of at least one inhibitor of the invention for the preparation of a pharmaceutical composition for the treatment of (for treating) a disease which is associated with an excess transport of hyaluronan across a lipid bilayer. The present invention also relates to a method for manufacturing a pharmaceutical composition comprising the steps of formulating the inhibitor defined herein in a pharmaceutically acceptable form.

A variety of documents is cited throughout this specification. The disclosure content of said documents (including any manufacturer's specifications, instructions etc.) is herewith incorporated by reference; however, there is no admission that any document cited is indeed prior art as to the present invention.

Hyaluronan is the major water binding component of the extracellular matrix. It is a very large glycosaminoglycan that is exported into the extracellular matrix by fibroblasts or epithelial cells, where it attracts water up to 99% of its own weight, swells to enormous volumes and displaces other resident macromolecules [1]. Typically, one molecule of hyaluronan with a molecular weight of 3.5 million Da totally occupies a sphere of about 440 nm [2]. Hyaluronan biosynthesis proceeds by alternate transfer of the precursor nucleotide sugars UDP-GlcA and UDP-GlcNac at the reducing end at the inner face of the plasma membrane [3-6]. The growing hyaluronan chain is synthesised within the cytoplasm and exported into the extracellular matrix where water attraction and swelling occurs. This mode of transmembrane transport was originally discovered in streptococci [7]. As the streptococcal hyaluronan transporter had structural and functional homology to human multidrug resistance transporters, we investigated hyaluronan exporters in human fibroblasts and identified MRP5 [8;9]. Our findings thus showed that two cellular processes are essential for the deposition of hyaluronan in the extracellular matrix, namely hyaluronan synthesis via the hyaluronan synthase in the cytosol and hyaluronan export through the plasma membrane via the MRP5 transporter.

In recent years it has become evident that cellular hyaluronan synthesis plays an important role in shedding and displacement of other components such as removal of antibodies or phagocytes from virulent Streptococci [10], detachment of fibroblasts during mitosis [11], tumour metastasis [12], as well as proteoglycan loss from osteoarthritic joints[13;14]. From all these observations, a new physiological function of hyaluronan can be postulated based on studies in several systems: Due to the enormous hydration volume, hyaluronan will replace any other components from its site of origin, when it extrudes from plasma membranes [1]. This concept may also apply for the rapid removal of mucus and adhering microorganisms from the bronchial epithelial surface that is pivotal for host defence.

The importance of hyaluronan for cellular behaviour had already been recognized for decades, but it was not until 1986 that the requirement for detachment in mitotic cell division was proven [11]. Hyaluronan was an adhesive cell surface component forming large coats around untransformed fibroblasts and smaller coats around transformed cells [15;16]. In humans, synthesis and degradation of hyaluronan is a very dynamic process. A total amount of hyaluronan of 34 mg is turned over in the circulation of an adult human daily [30;31]. The major origins of hyaluronan are joints, skin, eyes and intestine. The half-life in skin and joints is about 12 hours [32;33], in the anterior chamber of the eye it is 1-1.5 hours [34] and in the vitreous 70 days. The rapid turnover is surprising, because hyaluronan has been regarded as a structural component of the connective tissue.

### Cellular Functions

Fibroblasts are surrounded by a coat of hyaluronan that is lost upon transformation [16]. It modifies cell-cell aggregation [35], and cell-substratum adhesion [36]. Hyaluronan synthesis is coordinated with cell growth. Proliferating cells produce more hyaluronan than resting cells [37;38]. Synchronized fibroblasts show highest hyaluronan production during mitosis, because hyaluronan synthesis is required for detachment and mitosis of fibroblasts [11].

The function of hyaluronan on cellular behaviour appeared paradoxical. Hyaluronan synthesis is increased during cell migration [39], mitosis [11] and tumour invasion [19]. Overproduction of hyaluronan in the human tumour cell line HT1080 enhanced anchorage independent growth and tumorigenicity [40]. In contrast, hyaluronan promotes also cell adhesion [41], cell-cell aggregation [35] and inhibits proliferation [42;43].

A similar paradoxical situation applies for hyaluronidases in tumour progression. In some tumours, hyaluronidase treatment blocks lymph node invasion by tumour cell for T-cell lymphoma [44]. However, over expression of hyaluronidase correlates with disease progression in bladder, breast and prostate cancer [45-47].

A solution to this paradox was obtained from comparison of cellular behaviour of hyaluronan-deficient CHO cells and CHO cells transfected with the hyaluronan synthase. Surprisingly, hyaluronan synthesis reduced initial cell adhesion, migration, growth and the density at contact inhibition. All these apparent contradictions were combined into a model for the cellular functions of hyaluronan [29]. Thus hyaluronan serves as an adhesive component, when it is retained on the cell surface by intact CD44, and as a detachment factor, when it is released. Migration- and growth inhibitory effects of hyaluronan are mediated by proteolytic cleavage of CD44. The cellular function of hyaluronan could be an amplifier of cell surface proteases: when proteases are inactive, it amplifies the action of cellular adhesion factors by deposition and binding to receptors—when proteases are active, it amplifies cell detachment from the environment by activation of synthesis and shedding.

### Pathological Functions

Aberrant hyaluronan synthesis will lead to disturbances of cell behaviour and tissue integrity and hydration. For streptococci and pasteurella it serves as a non-antigenic disguising capsule in the infected host. The following overview almost reads like a survey of the major unsolved human diseases. The pathological disturbances are accompanied by a hyaluronan overproduction.

### Metastasis

Most malignant solid tumours contain elevated levels of hyaluronan. Enrichment of hyaluronan in tumours may be caused by increased production by tumours themselves or by induction in the surrounding stroma cells [19;58]. The mechanisms whereby hyaluronan-receptor interactions influence tumour cell behaviour are not clearly understood, and this is currently a very active area of investigation [12;29;59-61].

### Inflammation and Edema

Inflammation of various organs is often accompanied with an accumulation of hyaluronan. This can cause edema due to the osmotic activity and can lead to dysfunction of the organs. For example, hyaluronan accumulation in the rheumatoid joint impedes the flexibility of the joint [62]. Hyaluronan accumulation in rejected transplanted kidneys can cause edema and increased intracapsular pressure [63]. It also accumulates in pulmonary edema [64] and during myocardial infarction [65]. Large amounts of hyaluronan accumulate in demyelinated lesions in areas of multiple sclerosis [66] or ischemic stroke [67].

### Osteoarthritis

Osteoarthrosis is characterized by cartilage erosion, proteolysis of aggrecan and collagen and disturbed synthesis rates of aggrecan and hyaluronan by chondrocytes, hyaluronan overproduction being an early reaction. The destruction of joint cartilage is the major cause of human arthritic diseases, i.e., osteoarthrosis and rheumatoid arthritis. Chondrocytes represent only 5% of the tissue and are responsible for cartilage synthesis. It consists of two main components: the network of type II collagen, which provides the tensile strength and stiffness, and the large aggregating proteoglycan, aggrecan, which is responsible for the osmotic swelling capability and elasticity. Aggrecan decorates a backbone of hyaluronan that is anchored in the plasma membrane of chondrocytes at the hyaluronan synthase and further bound by the cell surface receptor CD44. The biosynthesis of hyaluronan and proteoglycans have different mechanisms and occur in different compartments [5]. Proteoglycans are synthesized in the Golgi and exocytosed by vesicles. Hyaluronan is polymerized at the inner side of plasma membranes [3-5] and exported by ABC-transporters [8]. Both components aggregate in the extracellular matrix [68] with up to 200 aggrecan molecules decorating one hyaluronan chain [69]. In healthy cartilage, the hyaluronan and aggrecan are synthesized and degraded at similar rates [70], whereas the turnover of collagens is much slower [71]. The proteoglycan residue is liberated from the hyaluronan binding region by aggrecanase [72]. Most of hyaluronan is removed by endocytosis through the CD44 receptor in healthy cartilage [73] or liberated into the environment in osteoarthritic cartilage [74]. Aggrecan leaves cartilage either as intact molecule or after proteolysis depending on the stimulus [75]. Aggregate formation is important from the physiological point of view, since it ensures the retention of aggrecan within the collagen network.

Key events in osteoarthritic cartilage are increased hyaluronan, decreased aggrecan synthesis [74;76] and proteolytic cleavage of collagen type II and aggrecan core protein [77;78]. For a long time, it was thought that proteolytic degradation of collagen and aggrecan was the primary event in cartilage breakdown. Many efforts to develop protease inhibitors led to compounds that were chondroprotective in vitro or in animal models [79-82], but results from clinical trials were equivocal [83;84]. Recently, we discovered that a variety of multidrug resistance inhibitors interferes with hyaluronan export from human cells, and their inhibition profile suggested that the multidrug resistance associated protein MRP5 could be a hyaluronan exporter [8]. The drugs prevented aggrecan loss from chondrocyte cell cultures, cartilage organ cultures and in an animal model of osteoarthrosis indicating that hyaluronan overproduction was involved in aggrecan loss [13]. We extended the analysis to the effects of the hyaluronan export inhibitors zaprinast, vardenafil and tadalafil to collagen degradation of II-1α activated bovine cartilage explants. The drugs normalized proteoglycan content and collagen degradation and reduced infiltration of degrading enzymes. Thus inhibition of hyaluronan export could be an appropriate target for therapeutic intervention in osteoarthrosis [14] (see also WO2005/013947).

Most injuries are followed by inflammation and hyaluronan overproduction that may lead to severe health problems. Excess hyaluronan production is observed after organ transplantation that may lead to tissue rejection, and after a heart infarct. It is also associated with alveolitis, pancreatitis, pulmonary or hepatic fibrosis, radiation induced inflammation, Crohn's disease, myocarditis, scleroderma, psoriasis, sarcoidosis [119a-135a]. Also many human tumors are characterized by an overproduction of hyaluronan such as melanoma [89a], mesothelioma [117a] or colon carcinoma [118a]. Because hyaluronan production is correlated with cell proliferation [86a], inhibition of hyaluronan transport will also reduce tumour growth. Overproduction of hyaluronan is also the cause of lump formation after contusion or insect bites, and therefore it will be possible to inhibit swelling by the inhibitors of hyaluronan transport.

Because aberrant hyaluronan synthesis will lead to disturbances of cell behaviour and tissue integrity and hydration, there is an ongoing need to provide inhibitors which are able to reduce the hyaluronan transport.

Thus, the technical problem underlying the present invention is to provide means and methods for treating and/or preventing diseases which are associated with an excess transport of hyaluronan across a lipid bilayer.

The solution to this technical problem is achieved by providing the embodiments characterized in the claims.

It must be noted that as used herein, the singular forms “a”, “an”, and “the”, include plural references unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

As mentioned before, overproduction of hyaluronan is a central problem in many diseases for example in ischemic or inflammatory edema or in arthritis. Also many human tumors are characterized by an overproduction of hyaluronan such as melanoma [89a], mesothelioma [117a] or colon carcinoma [118a]. Because hyaluronan production is corellated with cell proliferation [86a], inhibition of hyaluronan transport will also reduce tumor growth. Overproduction of hyaluronan is also the cause of lump formation after contusion or insect bites, therefore is will be possible to inhibit swelling by the inhibitors of hyaluronan transport. In fact, most injuries are followed by inflammation and hyaluronan overproduction that may lead to severe health problems such as organ transplantation and tissue rejection, hyaluronan production after a heart infarct, alveolitis, pancreatitis, pulmonary or hepatic fibrosis, radiation induced inflammation, Crohn's disease, myocarditis, scleroderma, psoriasis, sarcoidosis [119a-135a]. In particular, the unbalanced hyaluronan and proteoglycan synthesis by chondrocytes in arthritis appears to be the first biochemical event that eventually leads to complete joint destruction.

The present invention relates, in general, to inhibitors of the hyaluronan transport which are further specified herein below.

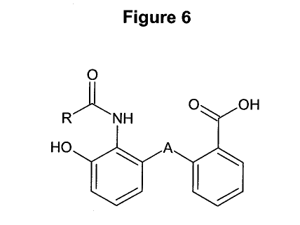

Starting from a lead structure (lead compound) which is depicted herein below (and also in FIG. 6) and which is exemplified by compound 86 (described herein), the present inventor found that increasing the hydrophobic moiety in No. 86 (leading to compound 96) led to an inhibitory compound with a fairly good IC50 value of 10 μM. Increasing the pKa of the carboxylic moiety from about 3, in No. 86, to about 8 by replacement with carbamate led to an inhibiting compound No. 97 with and IC₅₀ value of 30 μM. Similar hydroxamate based derivatives of aspirin have been shown to have superior properties [251]. Introducing a dimethylamino group in the o-position of the carboxylate is expected to reduce the pKa as well, and this compound No. 98 was slightly inhibiting with an IC50 value of 200 μM. Replacement of the carboxyl group by a sulfonylgroup reduces the pKa to about 1 and this leads to an inhibitory compound No 99 with IC₅₀˜80 μM.

From the above, one can conclude the following:

A suitable starting point for the design of the inhibitors of the invention is compound 86 which might be seen as a representative of the “lead compound” of the invention. Further representatives of the “lead compounds” of the invention which might be used as a starting compound for the design of the inhibitors of the present invention can be exemplified by the following formula:

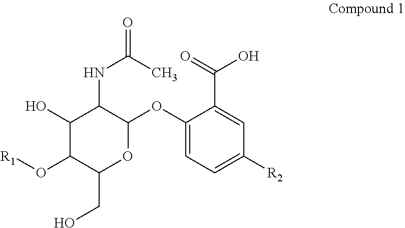

The lead compound or its representatives as defined above can be converted into an inhibitor of the invention for example by:
Introducing hydrophobic moieties, for example fluorobenzyl and chlorine (exemplified by compound 96)—further possible exchanges are exemplified in great detail herein below, and/or
converting the charged carboxyl group into a neutral group, or basic group, or more acidic group (exemplified by compound 97) e.g. amide —CO—NH2, Sulfonate —SO3H, Phosphonate —PO3H2, Phosphinate —PO2(R)OH—further possible exchanges are exemplified in great detail herein below, and/or
introducing a labile ester group that chemically modifies the protein to which the inhibitor is bound to (preferably MRP5)—exemplified by the following formula

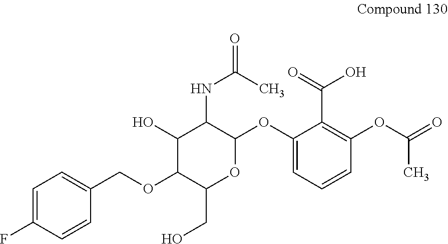

optionally, the carboxyl group of the lead compound, and/or other carboxyl groups which are contained in the compounds of the invention can be masked as an ester to prevent serious side effects due to stomach ulceration, a well known phenomenon for acidic NSARD. These esters are readily cleaved by serum or cytosolic esterases to form the active acidic compound. The alcohol that forms the ester can carry additional functional groups such in nitric oxide releasing aspirin derivatives [260].

It is preferred that the inhibitors of the present invention also obey the rule of 5 for “drugable” compounds:
There are not more than 5 H-bond donors (sum of OH and NH) in the molecule
There are no more than 10 H-bond acceptors (sum of N and O) in the molecule
The molecular weight does not exceed 500
Log P does not exceed 5
The PSA (Molecular polar surface area) does not exceed 150

These features can conveniently be calculated by the skilled person, for example when using the information contained in the free website http://www.molinspiration.com/cgi-bin/properties. However, even without the information provided by the referenced webpage, the skilled person is in a position to design an inhibitor which obeys the above stated well-recognized rules of 5 for “drugable” compounds.

It thus follows that the present invention relates to the lead compounddisclosed herein and also to compounds which are representatives of the lead compound of the invention and which are, or were converted into, inhibitors of the hyaluronan transport/expert. Such inhibitors are described herein in great detail. The capabilities of these inhibitors to inhibit/reduce the hyaluronan transport/export are derivable from the table below.

The present invention also relates to an inhibitor which is characterized by the following formula A, B, C, D, E or F:

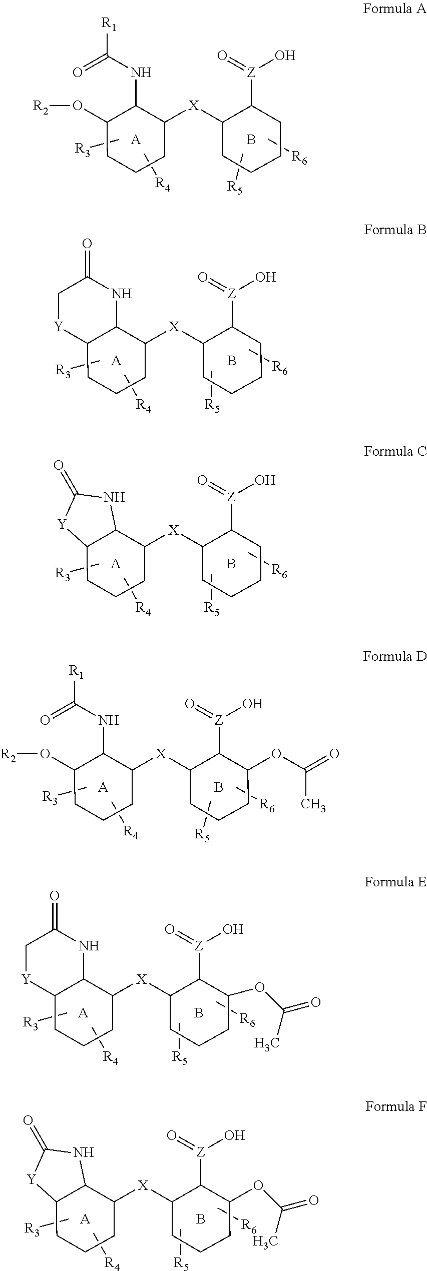

or a pharmaceutically acceptable salt thereof,
wherein
the ring systems A and B are independently selected from a monosaccharide, aryl (preferably phenyl), a heteroaryl ring or cycloalkyl (preferably cyclohexan), preferably with all substituents in equatorial configurations;
R1 is independently selected from alkyl (preferably C6-C12), a substituted or unsubstituted phenyl, preferably CH3;
R2 is H, alkyl (preferably C6 to C12), a carbohydrate in a glycosidic β-linkage, preferably H;
R3, R4, R5, and R6 are independently selected from (OH) hydroxy, alkyl (preferably C6 to C12), alkoxy (preferably C6 to C12), amino, alkylamino (preferably C6 to C12), halogen, cinnamylhydroxy, cinnamoyl, cinnamylamino, cinnamoylamino;
X is O, NH, alkylamino (NR), CO, S;
Z is C, sulfinate (S,) sulfonate (SO), phosphonate (O═P—OH), phosphinate (P—R, where R is alkyl or phenyl).

The term “benzylamino” refers to an amino group substituted with an benzyl group.

The term “benzoylamino” refers to an amino group substituted with a benzoyl group.

The term “cinnamylamino” refers to an amino group substituted with a cinnamyl group.

The term “cinnamoylamino” refers to an amino group substituted with a cinnamoyl group.

The terms “alkyl” and “alkylene” as used herein, whether used alone or as part of another group, refer to substituted or unsubstituted aliphatic hydrocarbon chains, the difference being that alkyl groups are monovalent (i.e., terminal) in nature whereas alkylene groups are divalent and typically serve as linkers. Both include, but are not limited to, straight and branched chains containing from 1 to about 12 carbon atoms, preferably 6 to 12 carbon atoms, unless explicitly specified otherwise. For example, methyl, ethyl, propyl, isopropyl, butyl, i-butyl and t-butyl are encompassed by the term “alkyl.” Specifically included within the definition of “alkyl” are those aliphatic hydrocarbon chains that are optionally substituted. Representative optional substituents include, but are not limited to, hydroxy, oxo (=0), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN, —OH, oxo (=0), and amino groups.

The carbon number as used in the definitions herein refers to carbon backbone and carbon branching, but does not include carbon atoms of the substituents, such as alkoxy substitutions and the like.

The term “alkenyl”, as used herein, whether used alone or as part of another group, refers to a substituted or unsubstituted aliphatic hydrocarbon chain and includes, but is not limited to, straight and branched chains having 2 to about 10 carbon atoms (unless explicitly specified otherwise) and containing at least one double bond. Preferably, the alkenyl moiety has 1 or 2 double bonds. Such alkenyl moieties can exist in the E or Z conformations and the compounds of this invention include both conformations. Specifically included within the definition of “alkenyl” are those aliphatic hydrocarbon chains that are optionally substituted. Representative optional substituents include, but are not limited to, hydroxy, acyloxy, alkoxy,’ amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Heteroatoms, such as O or S attached to an alkenyl should not be attached to a carbon atom that is bonded to a double bond. Preferred substituents include halogens, —CN, —OH, and amino groups

The term “alkynyl”, as used herein, whether used alone or as part of another group, refers to a substituted or unsubstituted aliphatic hydrocarbon chain and includes, but is not limited to, straight and branched chains having 2 to about 10 carbon atoms (unless explicitly 0 specified otherwise) and containing at least one triple bond. Preferably, the alkynyl moiety has about 2 to about 7 carbon atoms. In certain embodiments, the alkynyl can contain more than one triple bond and, in such cases, the alkynyl group must contain at least three carbon atoms. Specifically included within the definition of “alkynyl” are those aliphatic hydrocarbon chains that are optionally substituted. Representative optional substituents include, but are not limited to, hydroxy, \acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN, —OH, and amino groups Heteroatoms, such as O or S attached to an alkynyl should not be attached to the carbon that is bonded to a triple bond.

The term “cycloalkyl” as used herein, whether alone or as part of another group, refers to a substituted or unsubstituted alicyclic hydrocarbon group having 4 to about 7 carbon atoms, with 5 or 6 carbon atoms being preferred. “Cyclohexane” is even more preferred.

Specifically included within the definition of “cycloalkyl” are those alicyclic hydrocarbon groups that are optionally substituted. Representative optional substituents include, but are not limited to, hydroxy, oxo (=0), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl.

The term “aryl”, as used herein, whether used alone or as part of another group, is defined as a substituted or unsubstituted aromatic hydrocarbon ring group having 5 to about 10 carbon atoms (unless explicitly specified otherwise) with 5 to 7 carbon atoms being preferred. The “aryl” group can have a single ring or multiple condensed rings. The term“aryl” includes, but is not limited to phenyl, a-naphthyl, (3-naphthyl, biphenyl, anthryl, tetrahydronaphthyl, fluorenyl, indanyl, biphenylenyl, and acenaphthenyl. “Phenyl” is preferred.

Specifically included within the definition of “aryl” are those aromatic groups that are optionally substituted. In representative embodiments of the present invention, the, “aryl” groups are optionally substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. For example, the“aryl” groups can be optionally substituted with from 1 to 3 groups selected from Cl-C6 alkyl, Cl-C6 alkoxy, hydroxy, C3-C6 cycloalkyl, —(CH2)—C3-C6 cycloalkyl, halogen, Cl-C3 perfluoroalkyl, Cl-C3 perfluoroalkoxy, —(CH2)q-phenyl, and —O(CH2)q-phenyl. In these embodiments, the phenyl group of —(CH₂)q-phenyl and —O(CH2)q-phenyl can be optionally substituted with from 1 to 3 groups selected from Cl-C6 alkyl, Cl-C6 alkoxy, phenyl, halogen, trifluoromethyl or trifluoromethoxy. In other embodiments, phenyl groups of the present invention are optionally substituted with from 1 to 3 groups selected from Cl-C6 alkyl, Cl-C6 alkoxy, —(CH2) p-phenyl, halogen, trifluoromethyl or trifluoromethoxy. Preferred aryl groups include phenyl and naphthyl. Preferred substituents on the aryl groups herein include alkyl, alkoxy, halo, cyano, nitro, trihalomethyl, and thioalkoxy

As used herein, the term “heteroaryl”, whether used alone or as part of another group, is defined as a substituted or unsubstituted aromatic heterocyclic ring system (monocyclic or bicyclic). Heteroaryl groups can have, for example, from about 3 to about 50 carbon atoms (unless explicitly specified otherwise), with from about 4 about 10 being preferred. In some embodiments, heteroaryl groups are aromatic heterocyclic ring systems having about 4 to about 14 ring atoms and containing carbon atoms and 1,2, or 3 oxygen, nitrogen or sulfur heteroatoms. Representative heteroaryl groups are furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Specifically included within the definition of “heteroaryl” are those aromatic heterocyclic rings that are substituted with 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. In some embodiments of the present invention, the “heteroaryl” groups can be optionally substituted with from 1 to 3 groups selected from Cl-C6 alkyl, Cl-C6 alkoxy, hydroxy, C3-C6 cycloalkyl, —(CH2)-C3-C6 cycloalkyl, halogen, Cl-C3 perfluoroalkyl, Cl-C3 perfluoroalkoxy, —(CH2)q-phenyl, and —O(CH2)q-phenyl. In these embodiments, the phenyl group of —(CH2)q-phenyl and —O(CH2)q-phenyl can be optionally substituted with from 1 to 3 groups selected from Cl-C6 alkyl, Cl-C6 alkoxy, phenyl, halogen, trifluoromethyl or trifluoromethoxy. Preferred heterocycles of the present invention include substituted and unsubstituted furanyl, thiophenyl, benzofuranyl, benzothiophenyl, indolyl, pyrazolyl, oxazolyl, and fluorenyl.

As used herein, the term“phenylcycloalkyl”, whether used alone or as part of another group, refers to the group Ra-Rb—wherein Rb is an optionally substituted cyclized alkyl group having from about 3 to about 10 carbon atoms with from about 3 to about 6 being preferred and Ra is an optionally substituted phenyl group as described above. Preferred cycloalkyl groups are cyclopropyl, cyclobutyl, cyclopentyl or cyclohexyl. Examples of phenylcycloalkyl also include groups of formula: EMI9.1 wherein R7 and R8 are, independently, hydrogen, Cl-C6 alkyl, Cl-C6 alkoxy, hydroxy, —(CH2)q-phenyl, —O(CH2)q-phenyl, C3-C6 cycloalkyl, halogen, Cl-C3 perfluoroalkyl or Cl-C3 perfluoroalkoxy; m is from 1 to 4, and q=0-6.

The term “alkoxy” as used herein, refers to the group Ra-O—wherein Ra is an alkyl group as defined above. Specifically included within the definition of “alkoxy” are those alkoxy groups that are optionally substituted. Preferred substituents on alkoxy and thioalkoxy groups include halogens, —CN, —OH, and amino groups

The term “arylalkyl” or “aralkyl” refers to the group-Ra-Rb, where Ra is an alkyl group as defined above, substituted by Rb, an aryl group, as defined above. Aralkyl groups of the present invention are optionally substituted. Examples of arylalkyl moieties include, but are not limited to, benzyl, 1-phenylethyl, 2-phenylethyl, 3-phenylpropyl, 2-phenylpropyl and the like.

The term “halogen” or “halo” refers to chlorine, bromine, fluorine, and iodine.

The term “alkylamino” refers to groups having the formula selected from: (a) —(CH2)m-NH2, where m=1 to 12, preferably 6 to 12, (b) —NH—(CH2)n-NH2, where n=1 to 12, preferably 6 to 12, or (c) —NH—(C2H4NH)xC2H4NH2, where x=0 to 12, preferably 6 to 11.

The term “monosaccharide” includes trioses like glyceraldehyde or dihydroxyacetone; tetroses like erythrose, threose or erythrulose; pentoses like arabinose, lyxose, ribose, deoxyribose, xylose, ribulose and xylulose; hexoses like allose, altrose, galactose, glucose, gulose, idose, mannose, fructose, psicose, sorbose tagatose and talose; heptoses like mannoheptulose, sedoheptulose; octoses like octolose, 2-keto-3-deoxy-manno-octonate or nonoses like sialose.

The term “carbohydrate” includes monosaccharides as defined above, disaccharides, or oligosaccharides consisting of 1 to 10, preferably 1 to 3 monosaccharides.

In a preferred embodiment, A and B are phenyl, R1 is methyl, R2 is hydroxyl, and R3 are hydrogen, R4 is hydroxyl, R5 is hydrogen and R6 is cinnamylamino (Compound 96).

In a further preferred embodiment, A and B are phenyl, R1 is methyl, R2 is hydroxyl, and R3 are hydrogen, R4 is hydroxyl, R5 is hydrogen and R6 is chloro (Compound 97).

In a further preferred embodiment the lead compound was substituted in o-position of the carboxyl group with a dimethylamino group. This substitution is known to reduce the pKa of the acid (Compound 98).

In a further preferred embodiment the carboxyl group of the lead compound was substituted with a sulfonic acid to increase the acidity and it carries a chlorine in m-position to increase the lipophilicity (Compound 99).

In a further preferred embodiment N-actylglucosamine was substituted with 2-hydroxy-5-chloro-benzoic acid in 1-position and with a p-fluorobenzyl group in 4-position (Compound 110).

In a further preferred embodiment, A is glucosamine and B are phenyl, R1 is methyl, R2 is hydrogen, R3 is cinnamyl, R4 methoxy, and R5 and R6 are hydrogen (Compound 120).

In a further preferred embodiment N-actylglucosamine was substituted with 2-hydroxy-6-acetyl-benzoic acid in 1-position and with a p-fluorobenzyl group in 4-position (Compound 130).

In a preferred embodiment, the present invention relates to compounds 96, 97, 98, 99, 110, 120 and/or 130. The formulas of said compounds are depicted in the table below, including their inhibitory profile (IC50 for hyaluronan export).

| | | |
|---|---|---|
| Lead compound of the invention | 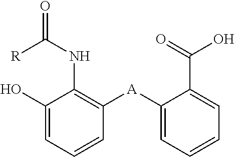 | |
| Representative of the lead compound of the invention (compound 1) R1 is for example p-Fluorobenzyl and R2 is for example chlorine (leading to compound 110) | 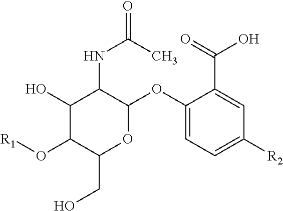 | |
| Representative of the lead compound of the invention (Compound 86) | 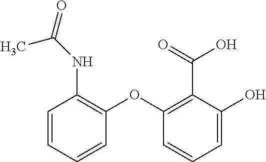 | no inhibitory activity |
| Compound 96 | 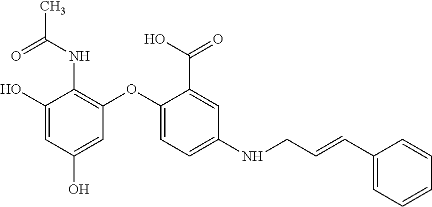 | IC 50 = 10 μM |
| Compound 97 | 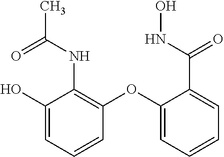 | IC 50 = 200 μM |
| Compound 98 | 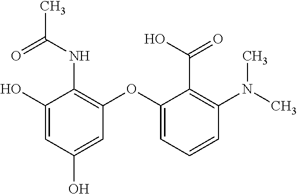 | IC 50 = 200 μM |
| Compound 99 | 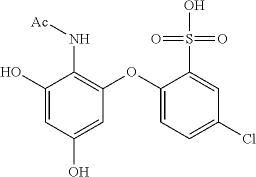 | IC 50 = 80 μM |
| Compound 110 | 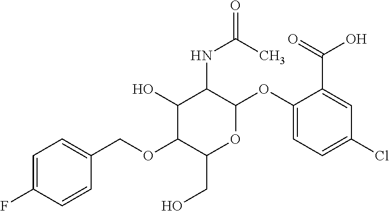 | |
| Compound 120 | 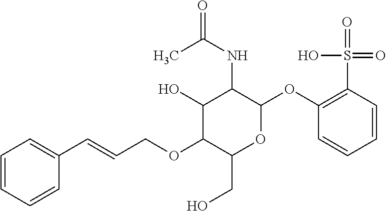 | |
| Compound 130 | 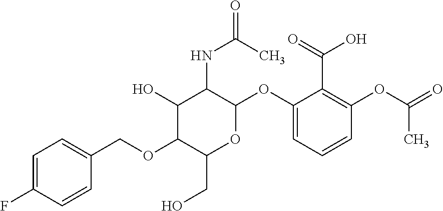 | |

The present invention also relates to an inhibitor based on compound 96, 97, 98, 99, 110, 120, and/or 130. “Based on” means chemically altered derivatives, which derivatives have, preferably, a comparable biological function when compared with one of the compounds selected from 96, 97, 98, 99, 110, 120, or 130. “Comparable biological function” means that the chemical derivatives of the invention are able to reduce the hyaluronan export with a deviation of the reducing activity in respect to one of the compounds selected form 96, 97, 98, 99, 110, 120, or 130 of not more than about 40%, 30%, 20%, 15%, 10%, 5%, 2.5%, 2% or 1%, for example under conditions which equate to or are identical with those set out in Example 1.

“Comparable biological function” does alternatively mean that the IC50 of the chemically altered derivatives of the invention deviates not more than about 40%, 30%, 20%, 15%, 10%, 5%, 2.5%, 2% or 1% from the IC50 of one of the compounds selected from 96, 97, 98, 99, 110, 120, or 130. WO2005/013947 discloses further suitable assays to evaluate the hyaluronan export.

The skilled person is able to justify which assay conditions/assays equate with the assay/conditions exemplified in the appended examples. The effect of the compounds of the invention hyaluronan transport/export reduction is thus determinable by the methods disclosed herein.

Also included are the pharmaceutically acceptable salts of the inhibitor(s) of the invention, including both organic and inorganic salts (e.g. with alkali and alkaline earth metals, ammonium, ethanolamine, diethanolamine and meglumine, chloride, hydrogen carbonate, phosphate, sulphate and acetate counterions). Appropriate pharmaceutically acceptable salts are well described in the pharmaceutical literature. In addition, some of these salts may form solvates with water or organic solvents such as ethanol. Such solvates are also included within the scope of this invention.

Furthermore, it has to be understood that the inhibitor(s) of the present invention, can be further modified to achieve (i) modified organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state).

From the inhibitory profile of certain drugs (see appended Example 9 of WO2005/013947) and inhibitory-experiments with MRP5-specific RNAi (see Example 10 of WO2005/013947) it is evident that MRP5 is the most likely hyaluronan transporter.

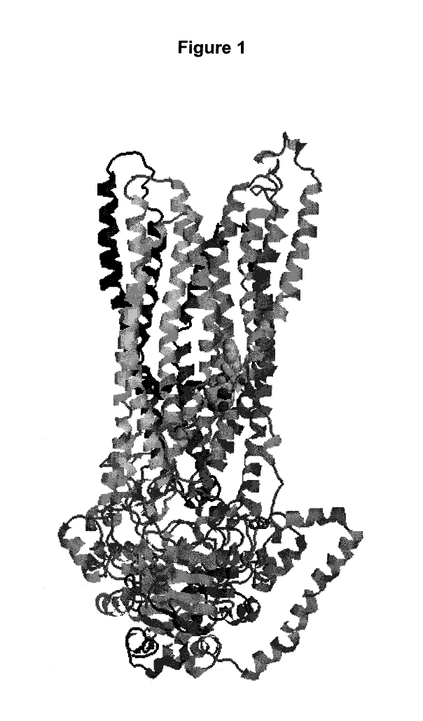

It is therefore preferred that the inhibitors of the present invention bind, preferably specifically, to the MRP5-transporter (see FIG. 1). MRP5 is an ABC-transporter which is described in great detail for example in WO2005/013947 (see for example the Examples 8 to 11). A comprehensive recent review on ABC transporters is [143a]. The web-site http://www.nutrigene.4t.com/humanabc.htm also contains valuable information.

In a further preferred embodiment, it is envisaged that the inhibitor of the invention at least reduces the hyaluronan transport rate about 5%, 10%, 20%, 30%, 40%, 50%, 60%, 70%, 80%, 90% or even 100% when compared to the transport rate that is achieved without the addition of said inhibitor. It is even more preferred that the hyaluronan transport rate of MRP5 is reduced. One specific screening assay for the hyaluronan transporter is based on the extrusion of labelled hyaluronan oligosaccharides from intact cells in monolayer culture. Said assay is further explained in WO2005/013947, particularly in the appended examples of said document (e.g Example 8 or Example 11). In such cases it is sufficient to analyse the effect of the inhibitor e.g. on a cell comprising MRP5, i.e. one compares the hyaluronan-transport before and after the addition of the inhibitor and thereby identifies inhibitors which reduce the transport-rate of hyaluronan across a lipid bilayer.

In a preferred embodiment of the uses or the methods of the present invention said inhibitor(s) specifically reduce(s) the transport of hyaluronan across a lipid bilayer mediated by MRP5. The term “specifically reduce(s)” used in accordance with the present invention means that the inhibitor specifically causes a reduction of the transport of hyaluronan as mediated by MRP5 but has no or essentially has no significant effect on other cellular proteins or enzymes.

The inhibitors can be discriminated by virtue of their binding to MRP5. Methods have been described that assay the binding of inhibitors to ABC transporters [92a;93a]. One specific screening assay for the hyaluronan transport as mediated by the ABC-transporter MRP5 is based on the extrusion of labeled hyaluronan oligosaccharides from intact cells in monolayer culture (see e.g. Example 11 of WO2005/013947). Alternatively, liposomes can be employed which encompass MRP5 in the lipid bilayer. For this assay, test-compounds like e.g. labeled hyaluronan oligosaccharides can be introduced into the cytosol of cells or into the liposomes. Because these test-compounds will normally not transverse the plasma membranes/lipid bilayer, they are introduced e.g. by osmotic lysis of pinocytotic vesicles according to a method that has already successfully been applied for the introduction of periodate oxidized nucleotide sugars [25a]. Alternatively, it is possible to introduce the test-compounds by other suitable methods like electro-chemical-poration; lipofection; bioballistics or microinjection (these methods are well-known in the art). Hyaluronan oligosaccharides are prepared from commercially available hyaluronan by digestion with hyaluronidase and sized fractionation by gel filtration as described [102a]. Appropriate oligosaccharide fractions having a length between 2 and 50 disaccharide units are labeled by incorporation of a biotin, radioactivity, or a fluorescent probe. These methods are routine published procedures [87a, 99a-101a, 103a]. For example the cells are seeded into multiwell microtiter plates to a density of at least 4×104 cells/cm². When the cells are attached to the plastic surface after a few hours, they are washed with phosphate buffered saline and incubated with the labeled hyaluronan dissolved in medium for osmotic lysis of pinocytotic vesicles (growth medium such as Dulbeccos medium containing 1 M sucrose, 50% poly(ethylene glycol)-1000) for at least 5 min up to several hours at 37° C. During this time the cells will pinocytose this hyperosmotic medium and the labeled hyaluronan. The above medium is substituted by a mixture of Dulbeccos medium and water (3:2) for 2 min. This causes the intracellular pinocytotic vesicles to lyse and to liberate the contents into the cytosol without damaging the cells. The cells can be subjected to this incubation sequence several times. The cells are washed thoroughly several times with phosphate buffered saline or growth medium to remove extracellular labeled hyaluronan and are then ready for the assay. They are incubated in growth medium containing the compound to be tested in different concentrations for several hours. During this time the labeled hyaluronan will be transported back into the medium. The amount of labeled hyaluronan oligosaccharide in the medium can be determined by a biotin-related assay, by radioactivity or by fluorescence intensity.

For medical treatment it is advantageous to use inhibitors that act in a reversible manner and do not block biochemical processes completely.

The inhibitors of the invention may be employed for the preparation of a pharmaceutical composition for the treatment of a disease which is associated with an excess transport of hyaluronan across a lipid bilayer.

The pharmaceutical composition of the present invention may optionally comprise a pharmaceutical carrier.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use.

Upon using the inhibitors of the present invention, it is possible to treat/ameliorate and/or prevent diseases which are associated with an excess transport of hyaluronan. The skilled person is well aware which specific diseases are characterized by an excess level of hyaluronan at the exterior of cells and, provided with the teaching and disclosure of the present invention can easily test for such an excess hyaluronan transport. Thus, it is possible to identify a subject at risk for a disease which is associated with an excess transport of hyaluronan across a lipid bilayer or to diagnose a disease which is associated with an excess transport of hyaluronan across a lipid bilayer. This can be diagnosed e.g., by isolating cells from an individual. Such cells can be collected from body fluids, skin, hair, biopsies and other sources as described herein elsewhere.

“A disease which is associated with an excess transport of hyaluronan” means in general that the disease is characterized by (is attended by) an abnormal production and/or by the abnormal presence of hyaluronan in cells, tissues and/or body fluids. This can be determined e.g., by isolating cells from an individual and or by evaluating the presence of hyaluronan otherwise (e.g. by help of antibodies directed against said molecule or by way of ELISA-assays which are able to evaluate the content of hyaluronan etc.). Such cells can be collected from body fluids, skin, hair, biopsies and other sources as described herein elsewhere.

An inhibitor of the present invention is preferably a compound which is characterized by a selection of one or more of the following capabilities: reduction of the overall transport of hyaluronan across a lipid-bilayer; reduction of the destruction of cartilage; maintenance of the actual state of destruction of cartilage; prevention of a further destruction of the cartilage etc; prevention of fluid accumulation in edema, prevention of tissue softening that is required for invasion of inflammatory cells or for metastasis, reduction of cell growth of tumors.

The term “excess transport” as used herein means that the transport of hyaluronan exceeds the transport level as compared with a normal/natural state of a comparable control-cell/subject. It has to be understood that in the context of the present invention, the terms “transport” and export” are used interchangeably.

The term “normal/natural state of a comparable control-cell/subject” means the transport-rate of hyaluronan in a control-cell which is preferably of the same nature as the test-cell (e.g. both cell are chondrocytes) but which is derived from a different source.

“A different source” includes e.g. a cell/tissue sample obtained from a healthy subject which does not suffer from a disease which is associated with an excess transport of hyaluronan across a lipid bilayer or a cell/tissue sample obtained from a distinct joint of the same subject wherein said different joint appears to be free from associated symptoms of a disease which is associated with an excess transport of hyaluronan across a lipid bilayer, e.g. arthritis. Assays and histological methods to classify a disease which is associated with an excess transport of hyaluronan across a lipid bilayer, e.g. arthritis (cartilage destruction associated with arthritis) are well-known to the skilled person (see for example WO2005/013947). However, even in cases where the inhibitor will not reduce the hyaluronan-transport across a lipid-bilayer to the normal/natural state of a comparable control-cell/subject but actually reduces the hyaluronan transport when compared to the transport rate before the addition of said inhibitor, it will be appreciated that said inhibitor has a beneficial effect.

The term “reduced” or “reducing” as used herein defines the reduction of the hyaluronan transport across a lipid bilayer, preferably to at least about the same level as compared to a normal/natural state of a comparable control-cell/subject.

Accordingly, it is preferred that the inhibitor of the invention at least reduces the hyaluronan transport rate about 5%, 10%, 20%, 30%, 40%, 50%, 60%, 70%, 80%, 90% or even 100% when compared to the transport rate that is achieved without the addition of said inhibitor. A suitable test system to measure the export/transport of hyaluronan is disclosed in the appended examples. Further test systems are disclosed in WO2005/013947.

It is preferred that the inhibitors of the invention have an IC50 between about 1 nanomolar and about 300 micromolar, more preferably between about 1 nanomolar and 1 micromolar and even more preferred between about 1 nanomolar and 100 nanomolar. “IC50” refers in this regard to the amount/concentration of the inhibitor which is necessary to reduce the hyaluronan transport to 50%, for example in a test assay exemplified in the appended examples or in any other suitable test system, for example test systems disclosed in WO2005/013947.

Assay for measuring the hyaluronan export/transport are disclosed in the appended examples. Further suitable assay are disclosed in WO2005/013947.

The present invention relates in one embodiment to the inhibitors as defined herein before as active compounds in a pharmaceutical composition.

It is also envisaged that the inhibitors of the present invention are used for treating (for the treatment) of a disease which is associated or characterized with an excess transport of hyaluronan across a lipid bilayer.

It is also envisaged that the inhibitors of the present invention (as defined herein before) are used for the preparation of a pharmaceutical composition for treating (for the treatment) of a disease which is associated or characterized with an excess transport of hyaluronan across a lipid bilayer.

The term “lipid bilayer” is well-known to the skilled person [91a] and denotes e.g. biological membranes or liposomes. Assay and test-systems which allow the determination of hyaluronan-transport across a lipid bilayer are explained in the appended examples. It will be understood that the term “capable of transporting hyaluronan across a lipid bilayer” defines in the context of cells or tissues comprising said cells, the transport of hyaluronan to the exterior of the cell (e.g. the extracellular milieu of the respective cell).

Examples for a “disease which is associated or characterized with an excess transport of hyaluronan across a lipid bilayer” are described herein in great detail and are furthermore known to the skilled person. Instructions, in the form of experimental tests or testable criteria, are therefore available from the patent document and from the common general knowledge (see for example WO2005/013947) allowing the skilled person to recognise which conditions fall within the functional definition and accordingly within the scope of the present invention.

Arthritis is, as mentioned before, accompanied with a loss of cartilage at the joint surface. The cartilage goes through different stages during pathogenesis. At first chondrocytes try to replace loss of cartilage by increased synthesis and proliferation; simultaneously lacunae of edema and increased water binding occurs which leads to softening of the cartilage matrix. At the second stage new cartilage production cannot compensate for the loss and at the third stage loss of cartilage is complete.

Thus, in a further embodiment of the medical uses and methods of the present invention said disease is characterized by degeneration and/or a destruction of cartilage.

The term “degeneration and/or destruction of cartilage” includes within the meaning of the present invention dysregulation of turnover and repair of joint tissue. The pathological features are focal areas of destruction of articular cartilage associated with hypertrophy of the subcondral bone, joint margin and capsule. The radiological changes include joint space narrowing, subchondral sclerosis and cysts, pain, loss of joint motion and disability.

The main hyaluronan producing cells in the body are fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, and epithelial cells. Chondrocytes represent only 5% of the tissue but they are responsible for synthesizing and controlling the matrix (including the hyaluronan production).

It is therefore envisaged that the inhibitors of the present invention are used for the treatment of (for treating) diseases which are associated or characterized with an excess transport of hyaluronan across a lipid bilayer of the cells mentioned above.

Diseases/conditions which may be treated with the inhibitors of the invention can be exemplified as follows: ischemic or inflammatory edema; tumors which are characterized by an overproduction of hyaluronan such as melanoma [89a], mesothelioma [117a] or colon carcinoma [118a]; lump formation after contusion or insect bites; injuries/conditions which are followed by inflammation and hyaluronan overproduction like heart infarct, alveolitis, pancreatitis, pulmonary or hepatic fibrosis, radiation induced inflammation, Crohn's disease, myocarditis, scleroderma, psoriasis, sarcoidosis [119a-135a]. Suppression of hyaluronan production can be beneficial for the diseases mentioned above.

Ischemic edema is caused by increased hyaluronan production around blood vessels that leads to vessel constriction and reduced oxygen supply. This is thought to be the main cause of death after a heart attack. Therefore immediate application of drugs inhibiting hyaluronan production will improve these conditions. Tissue necrosis will lead to inflammation with increased hyaluronan production that in turn causes edema. To break this vicious cycle inhibitors of hyaluronan transport will expand the choice for medical treatments.

Lump formation after contusion or insect bites can be life threatening, if for instance a bee-sting occurred in the throat. The swelling may suffocate the victim. Many metastatic tumors overproduce hyaluronan or stimulate the surrounding stroma to produce hyaluronan. The swollen tissue enables the metastatic tumor cells to easily invade the tissue. Therefore inhibition of hyaluronan transport will decrease the metastatic potential [136a-140a]. In addition hyaluronan production is also required for proliferation of fibroblasts [69a]. Therefore inhibition of hyaluronan transport will also reduce the growth of tumors.

The inhibitors of the present invention may also be used for the medical treatment of (for treating) arthritis. “Arthritis” includes, but is not limited to, osteoarthritis, (juvenile) chronic arthritis, rheumatoid arthritis, psoriatic arthritis, A. mutilans, septic arthritis, infectious arthritis and/or reactive arthritis. Thus, the term “arthritis” as used herein includes all forms of arthritis, e.g the primary or idiopathic form.

The secondary forms such as metabolic disorders such as ochronosis, acronmegaly, hemochromatosis and calcium crystal deposition and gout or apatite deposition; anatomic derangements such as slipped epiphysis, epiphysial dysplasias, Blount's disease, Legge-Perthe disease, congetial dislocation of the hip, leg length inequality, hypermobility syndromes; traumatic causes such as major joint trauma, fracture through a joint or osteonecrosis, joint surgery, chronic injury; any inflammatory arthropathy such as (juvenile) chronic arthritis, rheumatoid arthritis, psoriatic arthritis, A. mutilans, septic arthritis, infectious arthritis and/or reactive arthritis; joint abnormalities in thyroid diseases, diabetes melitus, hemophilia, amyloidosis, dialysis arthropathies, primary hyperlipidemias and xanthomatosis, Gaucher's disease, mucopolysaccharidosis; metabolic, regional and heritable bone and joint diseases such as osteoporosis, osteomalacia, renal bone diseases, algodystrophy/reflex sympathetic dystrophy syndrom, Paget's disease, hypertrophic osteoarthopathy, tumors of bone, heritable collagen disorders, hypermobility syndrome, joint dysplasias [96a]. The respective diseases as well as their symptoms are well-known to the skilled person and e.g. derivable from textbooks like Pschyrembel et al. or the like. Osteoarthritis (also known as osteoarthrosis or “non-inflammatory arthritis”) is a type of arthritis that is caused by the breakdown and eventual loss of the cartilage of one or more joints. Rheumatoid arthritis is an autoimmune disease that causes chronic inflammation of the joints. Rheumatoid arthritis can also cause inflammation of the tissue around the joints, as well as other organs in the body.

In a preferred embodiment said arthritis is osteoarthritis, (juvenile) chronic arthritis, rheumatoid arthritis, psoriatic arthritis, A. mutilans, septic arthritis, infectious arthritis and/or reactive arthritis.

The reduction which is achieved by the inhibitors of the present invention will also depend on the dosage and on the way of administration of the inhibitor. The dosage regimen utilizing the inhibitor of the present invention is therefore selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It has to be understood that in the context of the present invention, the term “at least one inhibitor” comprises at least one, at least two, at least three, at least four, at least five, at least six . . . etc. inhibitor(s) of the invention. It will be understood that the number of inhibitors which are used together (simultaneously or displaced) will be selected on a case to case basis in order to provide a suitable treatment for the cell/tissue/subject. In this context, “suitable” means that the treatment with the respective inhibitor(s) of the invention exerts a beneficial effect, e.g. it prevents, counters or arrests the progress of the condition (e.g. reduction of the overall transport of hyaluronan across a lipid-bilayer; reduction of the destruction of cartilage; maintenance of the actual state of destruction of cartilage; prevention of a further destruction of the cartilage etc.).

The inhibitors of the present invention can be applied prophylactically, for example with subjects that have or might have an enhanced individual risk factor such as obesity, heredity, for women after the menopause, osteoporosis, hypermobility, for persons with distorted joint shape or for persons with repetitive use of particular joint groups. Prophylactic treatment will be especially important for those diseases that will lead to an inflammation. Thus immediately after a heart attack it is expected the tissue necrosis may be a consequence. In this situation immediate prevention of increased hyaluronan production will reduce further complications. Similarly, organ transplantation will certainly increase hyaluronan production. Also in this case, reduction of hyaluronan transport will be beneficial.

Thus in a further embodiment of the medical uses of the present invention said inhibitor(s) is(are) to be administered prophylactically.

Alternatively, the inhibitors can by applied therapeutically as early as possible e.g. with respect to arthritis after diagnosis of joint insult to inhibit further destruction, to support self regeneration and restore joint function.

Thus, in another embodiment of the medical uses of the present invention said inhibitor(s) is(are) to be administered therapeutically.

The dosage regimen utilising the inhibitors or screened compounds(inhibitors) of the present invention is selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It is preferred that the inhibitors of the invention are used in a therapeutically effective amount/concentration, i.e. in an amount/concentration that is sufficient to exert its inhibitory effect. Said amount/concentration can be determined by the methods disclosed in the appended examples. It is envisaged that the therapeutically effective amount/concentration of inhibitor of the invention at least reduces the hyaluronan transport rate about 10%, 20%, 30%, 40%, 50%, 60%, 70%, 80%, 90% or even 100% when compared to the transport rate that is achieved without the addition of said inhibitor.

It is also envisaged that the inhibitors of the present invention are employed in co-therapy approaches, i.e. in co-administration with other medicaments or drugs, for example other drugs for preventing, treating or ameliorating arthritis which are known in the art (e.g. hyaluronan injections like Hyalgan® (Sanofi Pharmaceuticals), Orthovisc® (Anika Therapeutics) and SynVisc® (Biomatrix, now Genzyme), anti-inflammatory drugs and so on). It will be appreciated that these list of co-administered drugs is not limiting but severs as an example only. The skilled person is of course well-aware of suitable drugs which have a beneficial effect on arthritis and, therefore, might be useful when co-administered with the inhibitor(s) as described herein. With respect to the other diseases mentioned herein before (ischemic or inflammatory edema; tumors which are characterized by an overproduction of hyaluronan such as melanoma, mesothelioma or colon carcinoma; lump formation after contusion or insect bites; injuries/conditions which are followed by inflammation and hyaluronan overproduction like heart infarct, alveolitis, pancreatitis, pulmonary or hepatic fibrosis, radiation induced inflammation, Crohn's disease, myocarditis, scleroderma, psoriasis, sarcoidosis), it is envisaged that the inhibitor(s) of the present invention are co-administered e.g. together with cytostatics for the treatment of tumors and/or with antiinflammatory drugs for treatment of inflammations or inflammatory or ischemic edema and so on. Suitable compounds in this regard are well-known to the skilled artisan.

The present invention also relates to a method of preventing, ameliorating and/or treating the symptoms of a disease which is associated with an excess transport of hyaluronan across a lipid bilayer in a subject comprising administering at least one inhibitor as defined herein to the subject such that the disease which is associated with an excess transport of hyaluronan across a lipid bilayer is prevented, ameliorated and/or treated.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. The present invention is directed towards treating patients with medical conditions relating to a disease which is associated with an excess transport of hyaluronan across a lipid bilayer, e.g. arthritis. Accordingly, a treatment of the invention would involve preventing, inhibiting or relieving any medical condition related to a disease which is associated with an excess transport of hyaluronan across a lipid bilayer, e.g. arthritis. As mentioned elsewhere before, said arthritis is e.g. characterized by degeneration and/or a destruction of cartilage.

In the context of the present invention the term “subject” means an individual in need of a treatment of an affective disorder. Preferably, the subject is a mammalian, particularly preferred a human, a horse, a camel, a dog, a cat, a pig, a cow, a goat or a fowl.

The term “administered” means administration of a therapeutically effective dose of the inhibitors and/or test-compounds as disclosed herein. By “therapeutically effective amount” is meant a dose that produces the effects for which it is administered. The exact dose will depend on the purpose of the treatment, and will be ascertainable by one skilled in the art using known techniques.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments, i.e. it is also within the scope of the present invention to combine for example one of the already known drugs/treatments for arthritis (e.g. the injection of hyaluronan) with one or more of the inhibitors/test-compounds as defined herein.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or drug identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

It is also envisaged that the inhibitors of the present invention are employed in co-therapy approaches, i.e. in co-administration with other medicaments or drugs, for example other drugs for preventing, treating or ameliorating a disease which is associated with an excess transport of hyaluronan across a lipid bilayer, e.g. arthritis.

This disclosure may best be understood in conjunction with the accompanying drawings, incorporated herein by references. Furthermore, a better understanding of the present invention and of its many advantages will be had from the following examples, given by way of illustration and are not intended as limiting.

The figures show:

FIG. 1 3D model of MRP5 and an inhibitor of the invention

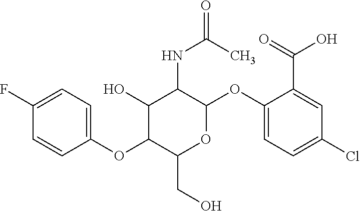

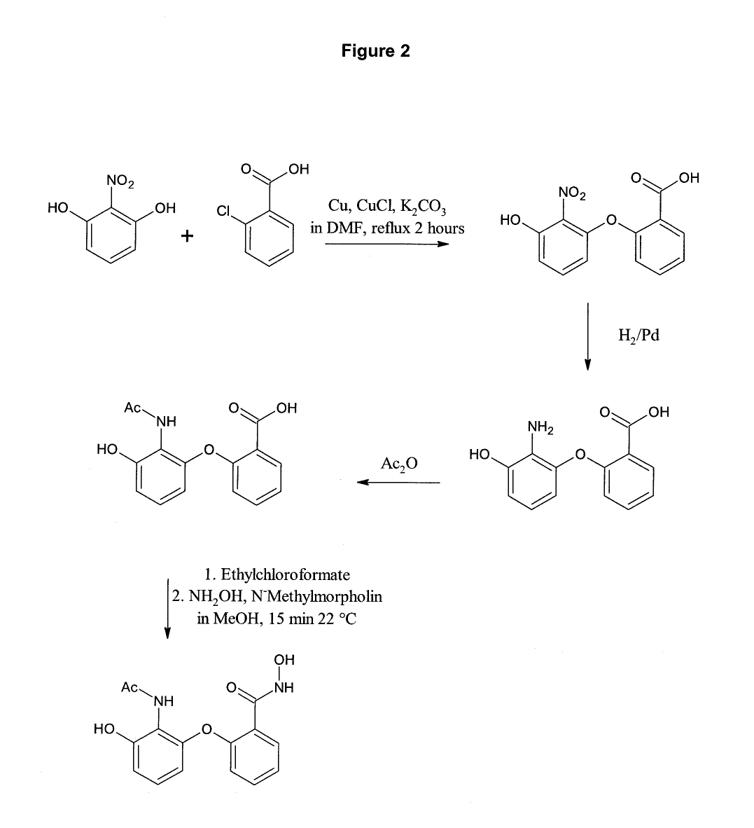

FIG. 2 Chemical synthesis of compound 86 and 97

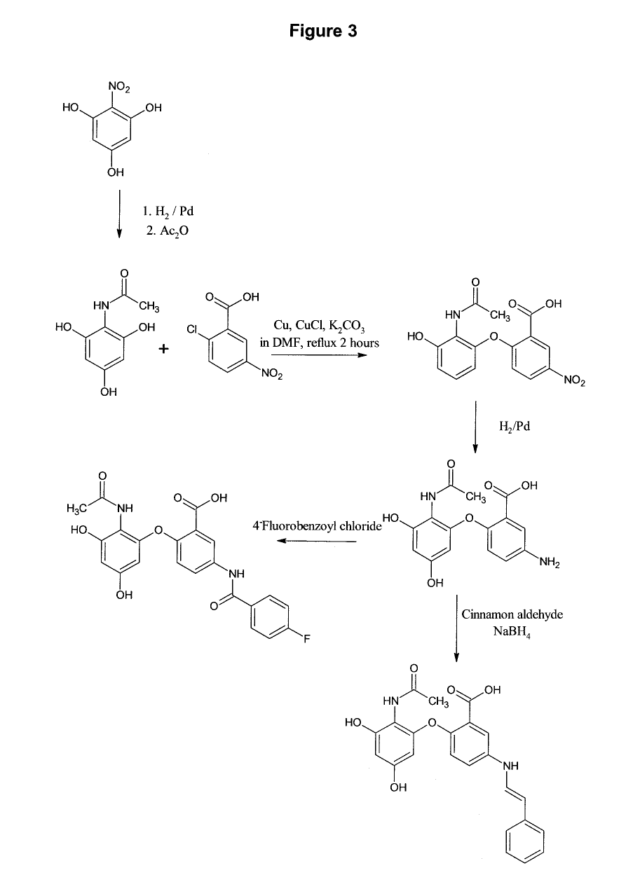

FIG. 3 Chemical synthesis of compound 89, 93 and 96

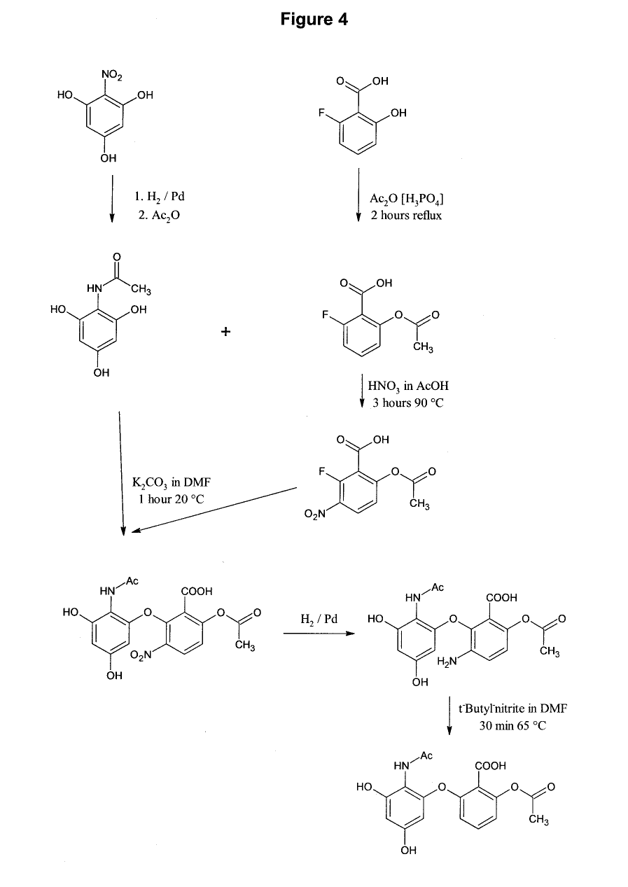

FIG. 4 Chemical synthesis of compound 95

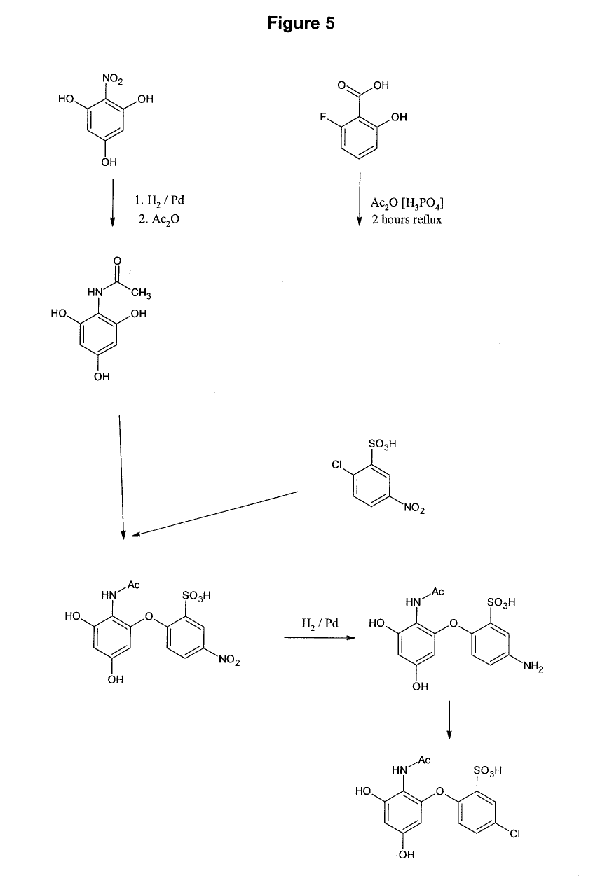

FIG. 5 Chemical synthesis of compound 99

FIG. 6 General structure of specific inhibitors of the hyaluronan transport/export

### EXAMPLES

The following examples illustrate the invention. These examples should not be construed as to limit the scope of this invention. The examples are included for purposes of illustration and the present invention is limited only by the claims.

### Example 1

### Assay for Hyaluronan Transport/Export Inhibitors in Fibroblast Cell Culture

Trypsinised fibroblasts were suspended in Dulbeccós medium at 10⁵ cells/ml and 100 μl aliquots were transferred to a 96 well microtiter plate. The first row received 200 μl of the suspension and 20 μl of the inhibitors of the invention dissolved in phosphate buffered saline at concentrations of 4 mM. A serial dilution of the inhibitors was established by transfer of 100 μl aliquots from the first row to the following rows. All experiments were performed in duplicates. The last row did not receive any inhibitor and served as control. The cells were incubated for 2 days at 37° C. and aliquots (5 and 20 μl) of the culture medium were used for measurement of the hyaluronan concentration in the cell culture medium by an ELISA [125]. Briefly, the wells of a 96 well Covalink-NH-microtiter plate (NUNC) were coated with 100 μl of a mixture of 100 mg/ml of hyaluronan (Healon®), 9.2 μg/ml of N-Hydroxysuccinimide-3-sulfonic acid and 615 μl/ml of 1-ethyl-3-(3-dimethylaminopropyl)-carbodiimide for 2 hours at room temperature and overnight at 4° C. The wells were washed three times with 2 M NaCl, 41 mM MgSO₄, 0.05% Tween-20 in 50 mM phosphate buffered saline pH 7.2 (buffer A) and once with 2 M NaCl, 41 mM MgSO₄, in phosphate buffered saline pH 7.2. Additional binding sites were blocked by incubation with 300 μl of 0.5% bovine serum albumin in phosphate buffered saline for 30 min at 37° C. Calibration of the assay was performed with standard concentrations of hyaluronan ranging from 15 ng/ml to 6000 ng/ml in equal volumes of culture medium as used for measurement of the cellular supernatants. A solution (50 μl) of the biotinylated hyaluronan binding fragment of aggrecan (Calbiochem) in 1.5 M NaCl, 0.3 M guanidinium hydrochloride, 0.08% bovine serum albumin 0.02% NaN₃ 25 mM phosphate buffer pH 7.0 was preincubated with 50 μl of the standard hyaluronan solutions or cellular supernatants for 1 hour at 37° C. The mixtures were transferred to the hyaluronan-coated test plate and incubated for 1 hour at 37° C. The microtiter plate was washed three times with buffer A and incubated with 100 μl/well of a solution of streptavidin-horseraddish-peroxidase (Amersham) at a dilution of 1:100 in phosphate buffered saline, 0.1% Tween-20 for 30 min at room temperature. The plate was washed five times with buffer A and the colour was developed by incubation with a 100 μl/well of a solution of 5 mg o-phenylenediamine and 5 μl 30% H₂O₂ in 10 ml of 0.1 M citrate-phosphate buffer pH 5.3 for 25 min at room temperature. The adsorption was read at 490 nm. The concentrations in the samples were calculated from a logarithmic regression curve of the hyaluronan standard solutions.

### Example 2

### Design of Compounds 95 to 99

Increasing the hydrophobic moiety in No. 96 (compared to compound 86) led to an inhibitory compound with a fairly good IC50 value of 10 μM. Increasing the pKa of the carboxylic moiety from about 3 in No. 86 to about 8 by replacement with carbamate led to a inhibiting compound No. 97 with and IC₅₀ value of 30 μM. Similar hydroxamate based derivatives of aspirin have been shown to have superior properties [251]. Introducing a dimethylamino group in the o-position of the carboxylate is expected to reduce the pKa as well, and this compound No. 98 was slightly inhibiting with a IC50 value of 200 μM. Replacement of the carboxyl group by a sulfonylgroup reduces the pKa to about 1 and this leads to an inhibitory compound No 99 with IC50˜80 μM.

### Example 3

### Chemical Synthesis of Compound 86 and 97

A mixture of 2.5 g 2-Nitroresorcinol (16 mMol), 2.5 g 2-Chlorobenzoic acid (16 mMol) 4.5 g K₂CO₃ (32 mMol), 50 mg copper, 50 mg CuCl in 20 ml dimethylformamid was refluxed for 3 hours. After cooling to room temperature, 20 ml of concentrated HCl and 200 ml of water was added, and the product was extracted with 200 of chloroform. The organic phase was dried over Na₂SO₄ and evaporated. The product was dissolved in 20 ml of methanol, 0.1 g of palladium (10% on charcoal) was added and hydrogenated in an H₂-atmosphere overnight at room temperature. The catalyst was removed by centrifugation, and the resulting amine was N-acetylated by addition of 0.8 g of acetic anhydride for 30 min at room temperature. The reagent was evaporated and last traces were removed by evaporation with toluene to obtain compound 86.

Compound 86 converted to the hydroxamic acid [259]. It (0.1 g; 0.35 mMol) was dissolved in 1.5 ml of methanol at 0° C. and 33 μl of ethylchloroformat and 38 μl of N-methyl morpholine was added. After 10 min, 0.35 mMol of hydroxylamine (freshly prepared from hydroxylamine hydrochloride and KOH in methanol) in 233 μl of methanol was added. After 15 min at room temperature, the solvent was evaporated and traces of the reactants were evaporated with toluene to obtain compound 97.

### Example 4

### Chemical Synthesis of Compounds 89, 93, and 96

Nitrophloroglucinol (1 g, 6.5 mMol) was dissolved in 10 ml of methanol and hydrogenated in a hydrogen atmosphere in the presence of 0.1 g of 10% Pd/C overnight at room temperature. The solvent was removed by evaporation and the residue was dissolved in 12 ml of dimethylformamide. 2-chlor-5-nitrobenzoic acid (1.2 g; 6 mMol), 1.7 g of K₂CO₃, 0.18 g of copper powder and 0.18 g of CuCl were added and the mixture was refluxed for 3 hours. After cooling to room temperature, 12 ml of concentrated HCl and 120 ml of water were added, and the product was extracted with 120 of ethylacetate. The organic phase was dried over Na₂SO₄ and evaporated. The product was dissolved in 12 ml of methanol, 0.1 g of palladium (10% ob charcoal) was added and hydrogenated in an H₂-atmosphere overnight at room temperature. The catalyst was removed by centrifugation, and the solvant was evaporated to obtain compound 89.

Compound 89 (2 mMol) was dissolved in 3 ml of methanol at 0° C. and 0.6 ml of 10 M NaOH and 0.5 μl of fluorbenzoylchlorid (4.2 mMol) was added. After 2.5 hours another portion of 1 ml of 10 M NaOH was added. After 30 min at room temperature, the mixture was acidified with concentrated hydrochloric acid, diluted with 30 ml of water and extracted with 20 ml of ethylacetate. The organic layer was dried and evaporated to obtain compound 93.

For reductive amination, compound 89 (1.33 mMol) was dissolved in 2 ml of methanol and 200 μl of cinnamon aldehyde (1.5 mMol) was added. NaBH₄ was added in 0.5 mM portions (19 mg each) every 3 hours, until the reaction was complete. The mixture was diluted with water, acidified and extracted with ether. The ether phase was dried, evaporated and compound 96 was obtained.

### Example 5

### Chemical Synthesis of Compound 95

6-Fluorosalicylic acid (0.5 g; 3.2 mMol) was acetylated with 2.5 ml of acetic anhydride in the presence of a drop of 85% phosphoric acid for 2 hours under reflux. Water (0.5 ml) was added and the solution was poured into 25 ml of cold water. The product was extracted with ether; the ether phase was dried and evaporated to obtain 6-fluoro-acetylsalicylic acid.

6-fluoro-acetylsalicylic acid was activated for the Ulmann condensation by nitration. It was dissolved in 5 ml of acetic acid and 0.2 ml of HNO₃ was added in 2 ml of acetic acid. The mixture was kept at 90° C. for 30 min, and 2 additional portions of HNO₃ were added every 30 min. The reaction mixture was chilled to room temperature, diluted with 30 ml of water and extracted with 20 ml of ether. The ether phase was dried and evaporated to obtain 5-nitro-6-fluoro-acetylsalicylic acid. 5-nitro-6-fluoro-acetylsalicylic acid (0.27 g; 1.1 mMol) was coupled to N-acetylaminophloro-glucinol (0.20 g) that had been prepared as described for compound 89 by addition of K₂CO₃ (0.5 g) in 2 ml of dimethylformamide for 1 hour at room temperature. 2 ml of concentrated HCl and 20 ml of water was added, and the product was extracted with 20 of chloroform. The organic phase was dried over Na₂SO₄ and evaporated. The product was dissolved in 5 ml of acetic acid; 0.1 g of palladium (10% on charcoal) was added and hydrogenated in an H₂-atmosphere overnight at room temperature. The catalyst was removed by centrifugation. The solvent was evaporated and the residue was dissolved in 4 ml of dimethylformamide. It was deaminated with t-butyl-nitrite (178 μl; 1.5 mMol) for 30 min at 65° C. The mixture was cooled, poured into 20 ml of cold HCl, extracted with 20 ml of ether, dried, and compound 95 was obtained by evaporation.

### Example 6

### Chemical Synthesis of Compound 99

2-Chloro-5-Nitrobenzenesulfonic acid Sodium Salt (250 mg 0.96 mMol) was coupled to acetylaminophloroglycinol (180 mg) in the presence of 0.28 g K₂CO, 0.04 g copper powder, 0.04 g CuCl, in 2 ml dimethylformamid under reflux for 1 hour. The mixture was diluted with 20 ml of water, acidified with 3 ml of concentrated sulfuric acid and extracted with 20 ml of ethylacetate. After evaporation of the solvent, the product was dissolved in 5 ml of methanol and reduced to the amine derivative by hydrogenation in a hydrogen atmosphere in the presence of, 0.1 g of palladium (10% on charcoal). The solvent was evaporated and the residue was dissolved in 2 ml of acetonitril. The compound was desaminated and chlorinated by addition of 178 μl t-Butyl-nitrite and 300 mg of CuCl at 65° C. for 35 min. The mixture was poured into cold sulfuric acid and the product was extracted with ethylacetate. The solvent was evaporated to obtain compound 99.

### Example 7

### Chemical Synthesis of Compound 98

Acetaminophloroglucinol was condensed in an Ullman reaction with an equimolar amount of 2-Nitro-6-fluoro-benzoic acid and the nitro group of the product was reduced with H₂/Pd in the presence of formic aldehyde as described in Journal of Organic Chemistry, 65, 7807-7813 (2000).

### Example 8

### Chemical Synthesis of Compound 110

Acetaminophloroglucinol was condensed in an Ullman reaction with an equimolar amount of 2-Chlor-5-nitrobenzoic acid and the nitro group of the product was reduced with H₂/Pd as described above. The amino group of the product (2 g) was acetylated by reaction with dimethylaminoacetyl chloride (1.8 g) in a solution of 3.8 ml triethylamine, 13 ml toluene and 15 ml dimethylformamide by refluxing overnight. The mixture was diluted with water, extracted with ether, dried, evaporated and purified by chromatography on silica gel.

### Example 9

### Chemical Synthesis of Compound 120

2-Acetamido-2-deoxy-4,6-O-isopropylidene-D-glucopyranose was prepared as described by A. Hasegawa in Carboh. Res. 29, 209 (1973). The product (33 g) was acetylated with 141 ml acetic anhydride in 150 pyridine ml for 12 hours at room temperature to give 2-acetamido-3-O-acetyl-4,6-isopropylidene-D-glucopyranose. The isopropylidene group was removed by treatment with 60% acetic acid at 50° C. for 3 hours to give 2-Acetamido-1,3-di-O-acetyl-glucopyranose. This product (20 g) was acetylated in 6-position by dissolving it in 75 ml dry pyridine and dropwise addition of acetic anhydride (6.65 g) in 15 ml of tetrahydrofuran. The solvents were evaporated and the product was purified by column chromatography on silica gel with chloroform/methanol (9:1). 2-Acetamido-1,3,6-tri-O-acetyl-glucopyranose (3.5 g, 10 mMol) was dissolved in 13 ml of dimethylsulfoxide and mixed with 16 ml of a 2 M solution of dimethylsulfinyl anion (prepared from 5 g NaH and 100 ml DMSO). After 10 min at room temperature 3.7 ml (30 Mol) of 4-fluorbenzylbromid was added. After 20 min the mixture was diluted with 100 ml of water and extracted with 50 ml of chloroform. The solvent was dried and evaporated. 2-Acetamido-4-Fluorobenzyl-3,6-di-O-acetyl-glucopyranosyl-bromid was obtained by treatment of 3 g of 2-Acetamido-4-Fluorobenzyl-1,3,6-tri-O-acetyl-glucopyranose in 15 ml of methylene chloride with 5.5 ml of 30% HBr in acetic acid for 2 hours at room temperature. The mixture was diluted with 100 ml of precooled methylene chloride and washed quickly with cold water, saturated NaHCO₃. The organic phase was dried and evaporated. The product was converted to the salicylic acid derivated by a modification of the method by SA Barker TH 1966, Supp 8 part II p 611. Briefly, 274 mg Methyl-5-chloro-2-hydroxybenzoate in 3 ml DMF was mixed with 0.6 ml of 2.5 M Butyllithium in Hexan and after 30 min 0.7 g of 2-Acetamido-4-Fluorobenzyl-3,6-di-O-acetyl-glucopyranosyl-bromid was added. The mixture was stirred for 14 h and poured into water. Deesterification was performed with 10 ml of water, 50 ml of methanol and 0.5 ml 10 M NaOH. The solution was neutralized by an ion exchanger avoiding acidification, as the product is acid labile.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

### REFERENCES

[1] Laurent, T. C. (1964) The interaction between polysaccharides and other macromolecules. The exclusion of molecules from hyaluronic acid gels and solutions. Biochem. J, 93, 106-112.
[2] Laurent, T. C. & Gergely, L. (1955) Light scattering studies on hyaluronic acid. J. Biol. Chem., 212, 325-333.
[3] Prehm, P. (1983) Synthesis of hyaluronate in differentiated teratocarcinoma cells. Mechanism of chain growth. Biochem. J., 211, 191-198.
[4] Prehm, P. (1983) Synthesis of hyaluronate in differentiated teratocarcinoma cells. Characterization of the synthase. Biochem. J., 211, 181-189.
[5] Prehm, P. (1984) Hyaluronate is synthesized at plasma membranes. Biochem. J., 220, 597-600.
[6] Prehm, P. (2006) Biosynthesis of Hyaluronan: Direction of Chain Elongation. Biochem. J., 398, 469-473.
[7] Ouskova, G., Spellerberg, B., & Prehm, P. (2004) Hyaluronan release from Streptococcus pyogenes: Export by an ABC transporter. Glycobiology, 14, 931-938.
[8] Prehm, P. & Schumacher, U. (2004) Inhibition of hyaluronan export from human fibroblasts by inhibitors of multidrug resistance transporters. Biochem. Pharmacol., 68, 1401-1410.
[9] Schulz, T., Schumacher, U., & Prehm, P. (2007) Hyaluronan export by the ABC-transporter MRP5 and its modulation by intracellular cGMP. J Biol. Chem., 282, 20999-21004.
[10] Nickel, V., Prehm, S., Lansing, M., Mausolf, A., Podbielski, A., Deutscher, J., & Prehm, P. (1998) An ectoprotein kinase of group C streptococci binds hyaluronan and regulates capsule formation. J. Biol. Chem., 273, 23668-23673.
[11] Brecht, M., Mayer, U., Schlosser, E., & Prehm, P. (1986) Increased hyaluronate synthesis is required for fibroblast detachment and mitosis. Biochem. J., 239, 445-450.
[12] Lüke, H. J. & Prehm, P. (1999) Synthesis and shedding of hyaluronan from plasma membranes of human fibroblasts and metastatic and non-metastatic melanoma cells. Biochem. J., 343, 71-75.
[13] Prehm, P. (2005) Inhibitors of hyaluronan export prevent proteoglycan loss from osteoarthritic cartilage. J. Rheumatol., 32, 690-696.
[14] Deiters, B. & Prehm, P. (2008) Inhibition of hyaluronan export reduces collagen degradation in IL-1 treated cartilage. Arthritis Res. Ther., 10, R8.
[15] Underhill, C. B. & Toole, B. P. (1979) Binding of hyaluronate to the surface of cultured cells. J. Cell Biol., 82, 475-484.
[16] Underhill, C. B. & Toole, B. P. (1982) Transformation-dependent loss of the hyaluronate-containing coats of cultured cells. J. Cell Physiol., 110, 123-128.
[19] Toole, B. P., Biswas, C., & Gross, J. (1979) Hyaluronate and invasiveness of the rabbit V2 carcinoma. Proc. Natl. Acad. Sci. U.S.A., 76, 6299-6303.
[29] Dube, B., Luke, H. J., Aumailley, M., & Prehm, P. (2001) Hyaluronan reduces migration and proliferation in CHO cells. Biochim. Biophys. Acta, 1538, 283-289.
[30] Fraser, J. R. & Laurent, T. C. (1989) Turnover and metabolism of hyaluronan. Ciba. Found. Symp., 143, 41-53.
[31] Lebel, L., Gabrielsson, J., Laurent, T. C., & Gerdin, B. (1994) Kinetics of circulating hyaluronan in humans. Eur. J. Clin. Invest., 24, 621-626.
[32] Reed, R. K., Laurent, T. C., & Taylor, A. E. (1990) Hyaluronan in prenodal lymph from skin: changes with lymph flow. Am. J. Physiol., 259, H1097-100.
[33] Coleman, P. J., Scott, D., Mason, R. M., & Levick, J. R. (2000) Role of hyaluronan chain length in buffering interstitial flow across synovium in rabbits. J. Physiol., 526, 425-434.
[34] Laurent, T. C., Dahl, L. B., & Lilja, K. (1993) Hyaluronan injected in the anterior chamber of the eye is catabolized in the liver. Exp. Eye Res., 57, 435-440.
[35] Underhill, C. B. (1982) Interaction of hyaluronate with the surface of simian virus40-transformed 3T3 cells: aggregation and binding studies. J. Cell Sci., 56, 177-189.
[36] Erickson, C. A. & Turley, E. A. (1983) Substrata formed by combination of extracellular matrix components alter neural crest cell migration. J. Cell Sci., 61, 299-323.
[37] Tomida, M., Koyama, H., & Ono, T. (1975) Induction of hyaluronic acid synthetase activity in rat fibroblasts by medium change of confluent cultures. J. Cell Physiol., 86, 121-130.
[38] Mian, N. (1986) Analysis of cell-growth-phase-related variations in hyaluronate synthase activity of isolated plasma-membrane fractions of cultured human skin fibroblasts. Biochem. J., 237, 333-342.
[39] Toole, B. P. (1991) Proteoglycans and hyaluronan in morphogenesis and differentiation. In Cell Biology of the Extracellular Matrix (Hay, E. D., ed), pp. 305-341. Plenum Press, New York.
[40] Kosaki, R., Watanabe, K., & Yamaguchi, Y. (1999) Overproduction of hyaluronan by expression of the hyaluronan synthase Has2 enhances anchorage-independent growth and tumorigenicity. Cancer Res., 59, 1141-1145.
[41] Miyake, K., Underhill, C. B., Lesley, J., & Kincade, P. W. (1990) Hyaluronate can function as a cell adhesion molecule and CD44 participates in hyaluronate recognition. J. Exp. Med., 172, 69-75.
[42] West, D. C. & Kumar, S. (1989) The effect of hyaluronate and its oligosaccharides on endothelial cell proliferation and monolayer integrity. Exp. Cell Res., 183, 179-196.
[43] Goldberg, R. L. & Toole, B. P. (1987) Hyaluronate inhibition of cell proliferation. Arthritis Rheum., 30, 769-778.
[44] Zahalka, M. A., Okon, E., Gosslar, U., Holzmann, B., & Naor, D. (1995) Lymph node (but not spleen) invasion by murine lymphoma is both CD44- and hyaluronate-dependent. J. Immunol., 154, 5345-5355.
[45] Lokeshwar, V. B., Obek, C., Soloway, M. S., & Block, N. L. (1997) Tumor-associated hyaluronic acid: A new sensitive and specific urine marker for bladder cancer. Cancer Res., 57, 773-777.
[46] Bertrand, P., Girard, N., Duval, C., D'Anjou, J., Chauzy, C., Menard, J. F., & Delpech, B. (1997) Increased hyaluronidase levels in breast tumor metastases. Int. J. Cancer, 73, 327-331.
[47] Lokeshwar, V. B., Lokeshwar, B. L., Pham, H. T., & Block, N. L. (1996) Association of elevated levels of hyaluronidase, a matrix-degrading enzyme, with prostate cancer progression. Cancer Res., 56, 651-657.
[58] Knudson, W., Biswas, C., & Toole, B. P. (1984) Interactions between human tumor cells and fibroblasts stimulate hyaluronate synthesis. Proc. Natl. Acad. Sci. U.S.A., 81, 6767-6771.
[59] Toole, B. P. (2004) Hyaluronan: from extracellular glue to pericellular cue. Nat. Rev. Cancer, 4, 528-539.
[60] Jojovic, M., Delpech, B., Prehm, P., & Schumacher, U. (2002) Expression of hyaluronate and hyaluronate synthase in human primary tumours and their metastases in scid mice. Cancer Lett., 188, 181-189.
[61] Monz, K., Maas-Kück, K., Schumacher, U., Schulz, T., Schneider, S. W., & Prehm, P. (2008) Inhibition of hyaluronan export attenuates cell migration and metastasis of human melanoma. submitted.
[62] Engström-Laurent, A. (1997) Hyaluronan in joint disease. J. Intern. Med., 242, 57-60.
[63] Hallgren, R., Gerdin, B., & Tufveson, G. (1990) Hyaluronic acid accumulation and redistribution in rejecting rat kidney graft. Relationship oto the transplantation edema. J. Exp. Med., 171, 2063-2076.
[64] Nettelbladt, O., Tengblad, A., & Hallgren, R. (1989) Lung accumulation of hyaluronan parallels pulmonary edema in experimental alveolitis. Am. J. Physiol., 257, L379-L384.
[65] Waldenstrom, A., Martinussen, H. J., Gerdin, B., & Hallgren, R. (1991) Accumulation of hyaluronan and tissue edema in experimental myocardial infarction. J. Clin. Invest., 88, 1622-1628.
[66] Back, S. A., Tuohy, T. M., Chen, H., Wallingford, N., Craig, A., Struve, J., Luo, N. L., Banine, F., Liu, Y., Chang, A., Trapp, B. D., Bebo, B. F., Rao, M. S., & Sherman, L. S. (2005) Hyaluronan accumulates in demyelinated lesions and inhibits oligodendrocyte progenitor maturation. Nat. Med.
[67] Al'qteishat, A., Gaffney, J., Krupinski, J., Rubio, F., West, D., Kumar, S., Kumar, P., Mitsios, N., & Slevin, M. (2006) Changes in hyaluronan production and metabolism following ischaemic stroke in man. Brain., 129, 2158-2176.
[68] Prehm, P. (1986) Mechanism, localization, and inhibition of hyaluronate synthesis. In Articular cartilage biochemistry (Kuettner, K., ed), pp. 81-91. Raven Press, New York.
[69] Tang, L. H., Buckwalter, J. A., & Rosenberg, L. C. (1996) Effect of link protein concentration on articular cartilage proteoglycan aggregation. J. Orthop. Res., 14, 334-339.
[70] Morales, T. I. & Hascall, V. C. (1988) Correlated metabolism of proteoglycans and hyaluronic acid in bovine cartilage organ cultures. J. Biol. Chem., 263, 3632-3638.
[71] Maroudas, A., Palla, G., & Gilav, E. (1992) Racemization of aspartic acid in human articular cartilage. Connect. Tissue Res., 28, 161-169.
[72] Embry, J. J. & Knudson, W. (2003) G1 domain of aggrecan cointernalizes with hyaluronan via a CD44-mediated mechanism in bovine articular chondrocytes. Arthritis Rheum., 48, 3431-3441.
[73] Hua, Q., Knudson, C. B., & Knudson, W. (1993) Internalization of hyaluronan by chondrocytes occurs via receptor-mediated endocytosis. J. Cell Sci., 106, 365-375.
[74] D'Souza, A. L., Masuda, K., Otten, L. M., Nishida, Y., Knudson, W., & Thonar, E. J. (2000) Differential Effects of Interleukin-1 on Hyaluronan and Proteoglycan Metabolism in Two Compartments of the Matrix Formed by Articular Chondrocytes Maintained in Alginate. Arch. Biochem. Biophys., 374, 59-65.
[75] Sztrolovics, R., White, R. J., Roughley, P. J., & Mort, J. S. (2002) The mechanism of aggrecan release from cartilage differs with tissue origin and the agent used to stimulate catabolism. Biochem. J., 362, 465-472.
[76] Nishida, Y., D'Souza, A. L., Thonar, E. J., & Knudson, W. (2000) Stimulation of hyaluronan metabolism by interleukin-1alpha in human articular cartilage. Arthritis Rheum., 43, 1315-1326.
[77] Smith, R. L. (1999) Degradative enzymes in osteoarthritis. Front Biosci., 4, D704-D712.
[78] Poole, A. R., Nelson, F., Dahlberg, L., Tchetina, E., Kobayashi, M., Yasuda, T., Layerty, S., Squires, G., Kojima, T., Wu, W., & Billinghurst, R. C. (2003) Proteolysis of the collagen fibril in osteoarthritis. Biochem. Soc. Symp., 70, 115-123.
[79] Bottomley, K. M., Borkakoti, N., Bradshaw, D., Brown, P. A., Broadhurst, M. J., Budd, J. M., Elliott, L., Eyers, P., Hallam, T. J., Handa, B. K., Hill, C. H., James, M., Lahm, H. W., Lawton, G., Merritt, J. E., Nixon, J. S., Rothlisberger, U., Whittle, A., & Johnson, W. H. (1997) Inhibition of bovine nasal cartilage degradation by selective matrix metalloproteinase inhibitors. Biochem. J., 323, 483-488.
[80] Caputo, C. B., Sygowski, L. A., Wolanin, D. J., Patton, S. P., Caccese, R. G., Shaw, A., Roberts, R. A., & DiPasquale, G. (1987) Effect of synthetic metalloprotease inhibitors on cartilage autolysis in vitro. J. Pharmacol. Exp. Ther., 240, 460-465.
[81] Cawston, T., Plumpton, T., Curry, V., Ellis, A., & Powell, L. (1994) Role of TIMP and MMP inhibition in preventing connective tissue breakdown. Ann. N. Y. Acad. Sci., 732, 75-83.
[82] Seed, M. P., Thomson, T. A., & Gardner, C. R. (1991) Investigation of the role of metalloproteinases in recombinant human interleukin-1 beta-induced degradation of rat femoral head cartilage. Drugs Exp. Clin. Res., 17, 355-361
[83] Greenwald, R. A. (1999) Thirty-six years in the clinic without an MMP inhibitor. What hath collagenase wrought? Ann. N. Y. Acad. Sci., 878, 413-419.
[84] Elliott, S. & Cawston, T. (2001) The clinical potential of matrix metalloproteinase inhibitors in the rheumatic disorders. Drugs & Aging, 18, 87-99.
[121] Pilcher, H. (2004) Medicinal leeches: stuck on you. Nature., 432, 10-11.
[122] Michalsen, A., Klotz, S., Ludtke, R., Moebus, S., Spahn, G., & Dobos, G. J. (2003) Effectiveness of leech therapy in osteoarthritis of the knee: a randomized, controlled trial. Ann. Intern. Med., 139, 724-730.
[123] Michalsen, A., Moebus, S., Spahn, G., Esch, T., Langhorst, J., & Dobos, G. J. (2002) Leech therapy for symptomatic treatment of knee osteoarthritis: results and implications of a pilot study. Altern. Ther. Health Med., 8, 84-88.
[124] Michalsen, A., Deuse, U., Esch, T., Dobos, G., & Moebus, S. (2001) Effect of leeches therapy (Hirudo medicinalis) in painful osteoarthritis of the knee: a pilot study. Ann. Rheum. Dis., 60, 986.
[125] Stern, M. & Stern, R. (1992) An ELISA-like assay for hyaluronidase and hyaluronidaseinhibitors. Matrix, 12, 397-403.
[127] Zinke, T. & Würker, W. Ueber Dinitrophenylpyridiniumchlorid und dessen Umwandlungsproducte (2. Mittheilung.). Ueber Dinitrophenylpyridiniumchlorid und dessen Umwandlungsproducte. Liebigs Ann. Chem. 338[121], 141. 1904.
[128] König, W. Ober eine neue, vom Pyridin deivierende Klasse von Farbstoffen. J. Prakt. Chem. 69, 105-137. 1904.
[129] Garmaise, D. L., Paris, G. Y., Komlossy, J., & McCrae, R. C. Anthelmintic Quaternary Salts: IV. Aminopentadienylideneammonium Salts. J. Am. Chem. Soc. 12, 806. 1969.
[130] Reichardt, C., Harms, K., Kinzel, M., Schafer, G., Stein, J., & Wocadlo, S. Chiral Polymethine Dyes, IV. Synthesis, absolute configuration, spectroscopic and chiroptical properties of chiral tri- and pentamethiunium cyanine dyes with 1,2,3,4-tetrahydro-6-methylquinolyl end groups. Liebigs Ann. Chem. 317-327. 1995.
[133] Sagawa, T., Tobata, H., & Ihara, H. (2004) Exciton interactions in cyanine dye—hyaluronic acid (HA) complex: reversible and biphasic molecular switching of chromophores induced by random coil-to-double-helix phase transition of HA. Chem. Commun. (Camb.), 2090-2091.
[134] Santos, H. A., Manzanares, J. A., Murtomaki, L., & Kontturi, K. (2007) Thermodynamic analysis of binding between drugs and glycosaminoglycans by isothermal titration calorimetry and fluorescence spectroscopy. Eur. J. Pharm. Sci., 32, 105-114.
[135] Lopes, S. & Castanho, M. A. (2004) Does aliphatic chain length influence carbocyanines' orientation in supported lipid multilayers? J Fluoresc., 14, 281-287.
[251] Dooley, C. M., Devocelle, M., McLoughlin, B., Nolan, K. B., Fitzgerald, D. J., & Sharkey, C. T. (2003) A novel family of hydroxamate-based acylating inhibitors of cyclooxygenase. Mol. Pharmacol., 63, 450-455.
[252] Hack, N., Carey, F., & Crawford, N. (1984) The inhibition of platelet cyclo-oxygenase by aspirin is associated with the acetylation of a 72 kDa polypeptide in the intracellular membranes. Biochem. J, 223, 105-111.
[253] Lecomte, M., Laneuville, O., Ji, C., DeWitt, D. L., & Smith, W. L. (1994) Acetylation of human prostaglandin endoperoxide synthase-2 (cyclooxygenase-2) by aspirin. J Biol. Chem., 269, 13207-13215.
[259] Reddy, A. S., Kumar, M. S., & Reddy, G. R. (2000) A convenient method for the preparation of hydroxamic acid. Tetrahydron Letters, 41, 6285-6288.
[260] Gresele, P. & Momi, S. (2006) Pharmacologic profile and therapeutic potential of NCX 4016, a nitric oxide-releasing aspirin, for cardiovascular disorders. Cardiovasc. Drug Rev., 24, 148-168.
[25a] Prehm, P. (1985) Inhibition of hyaluronate synthesis. Biochem. J., 225, 699-705.
[69a] Brecht, M., Mayer, U., Schlosser, E., & Prehm, P. (1986) Increased hyaluronate synthesis is required for fibroblast detachment and mitosis. Biochem. J., 239, 445-450.
[86a] Dube, B., Luke, H. J., Aumailley, M., & Prehm, P. (2001) Hyaluronan reduces migration and proliferation in CHO cells. Biochim. Biophys. Acta, 1538, 283-289.
[87a] Stern, M. & Stern, R. (1992) An ELISA-like assay for hyaluronidase and hyaluronidaseinhibitors. Matrix, 12, 397-403.
[89a] Lüke, H. J. & Prehm, P. (1999) Synthesis and shedding of hyaluronan from plasma membranes of human fibroblasts and metastatic and non-metastatic melanoma cells. Biochem. J., 343, 71-75.
[91a] Alberts, B., Bray, D., Lewis, J., Raff, M., Roberts, K., & Watson, J. D. (2002) Molecular Biology of the Cell, 3 edn. Garland, N.Y.
[92a] Stein, W. D. (1997) Kinetics of the multidrug transporter (P-glycoprotein) and its reversal. Physiol Rev., 77, 545-590.
[93a] Twentyman, P. R., Rhodes, T., & Rayner, S. (1994) A comparison of rhodamine 123 accumulation and efflux in cells with P-glycoprotein-mediated and MRP-associated multidrug resistance phenotypes. Eur. J. Cancer, 30A, 1360-1369.
[96a] Cooper, C. (1998) Osteoarthritis and related disorders. Epidemiology. In Rheumatology (Klippel, J. H. & Dieppe, P. A., eds), pp. 2.1-2.8. Mosby, London.
[99a] Stern, M. & Stern, R. (1992) An ELISA-like assay for hyaluronidase and hyaluronidaseinhibitors. Matrix, 12, 397-403.
[100a] de Belder, A. N. & Wik, K. O. (1975) Preparation and properties of fluorescein-labelled hyaluronate. Carbohydr. Res., 44, 251-257.
[101a] Rao, C. M., Jilani, A., Swarnakar, S., Deb, T. B., & Datta, K. (1996) A method to radioiodinate hyaluronic acid and its use as a probe to detect hyaluronic acid-binding proteins. J. Biol. Chem., 255, 7218-7224.
[102a] Termeer, C. C., Hennies, J., Voith, U., Ahrens, T., Weiss, M., Prehm, P., & Simon, J. C. (2000) Oligosaccharides of hyaluronan are potent activators of dendritic cells. J. Immunol., 165, 1863-1870.
[103a] Prehm, P. & Scheid, A. (1978) Sensitive method for the analysis of carbohydrates by gas chromatography of 3H-labeled alditol acetates. J. Chromatogr., 166, 461-467.
[117a] Asplund, T., Versnel, M. A., Laurent, T. C., & Heldin, P. (1993) Human mesothelioma cells produce factors that stimulate the production of hyaluronan by mesothelial cells and fibroblasts. Cancer Res., 53, 388-392.
[118a] Jacobson, A., Rahmanian, M., Rubin, K., & Heldin, P. (2002) Expression of hyaluronan synthase 2 or hyaluronidase 1 differentially affect the growth rate of transplantable colon carcinoma cell tumors. Int. J. Cancer, 102, 212-219.
[119a] Tufveson, G., Hallgren, R., Johnsson, C., & Wahlberg, J. Use of hyaluronidase in treatment of interstitial edema from organ grafts. Tufveson, Gunnar, Hallgren, Roger, Johnsson, Cecilia, and Wahlberg, Jan. [WO 9808538],
[120a] Engstrom Laurent, A., Feltelius, N., Hallgren, R., & Wasteson, A. (1985) Raised serum hyaluronate levels in scleroderma: an effect of growth factor induced activation of connective tissuecells? Ann. Rheum. Dis., 44, 614-620.
[121a] Lundin, A., Engstrom Laurent, A., Hallgren, R., & Michaelsson, G. (1985) Circulating hyaluronate in psoriasis. Br. J. Dermatol., 112, 663-671.
[122a] Hallgren, R., Eklund, A., Engstrom Laurent, A., & Schmekel, B. (1985) Hyaluronate in bronchoalveolar lavage fluid: a new markerin sarcoidosis reflecting pulmonary disease. Br. Med. J. Clin. Res. Ed., 290, 1778-1781.
[123a] Eklund, A., Hallgren, R., Blaschke, E., Engstrom Laurent, A. P.-U., & Svane, B. (1987) Hyaluronate in bronchoalveolar lavage fluid in sarcoidosis and its relationship to alveolar cell populations. Eur. J. Respir. Dis., 71, 30-36.
[124a] Nettelbladt, O. & Hallgren, R. (1989) Hyaluronan (hyaluronic acid) in bronchoalveolar lavage fluidduring the development of bleomycin-induced alveolitis in therat. Am. Rev. Respir. Dis., 140, 1028-1032.
[125a] Nettelbladt, O., Lundberg, K., Tengblad, A., & Hallgren, R. (1990) Accumulation of hyaluronan in bronchoalveolar lavage fluid is independent of iron-, complement- and granulocyte-depletionin bleomycin-induced alveolitis in the rat. Eur. Respir. J., 3, 765-771.
[126a] Hallgren, R., Gerdin, B., Tengblad, A., & Tufveson, G. (1990) Accumulation of hyaluronan (hyaluronic acid) in myocardial interstitial tissue parallels development of transplantation edema in heart allografts in rats. J. Clin. Invest, 85, 668-673.
[127a] Nettelbladt, O., Scheynius, A., Bergh, J., Tengblad, A., & Hallgren, R. (1991) Alveolar accumulation of hyaluronan and alveolar cellular response in bleomycin-induced alveolitis. Eur. Respir. J., 4, 407-414.
[128a] Bjermer, L., Hallgren, R., Nilsson, K., Franzen, L., Sandstrom, T. S.-B., & Henriksson, R. (1992) Radiation-induced increase in hyaluronan and fibronectin inbronchoalveolar lavage fluid from breast cancer patients is suppressed by smoking. Eur. Respir. J., 5, 785-790.
[129a] Ahrenstedt, O., Knutson, L., Hallgren, R., & Gerdin, B. (1992) Increased luminal release of hyaluronan in uninvolved jejunum in active Crohn's disease but not in inactive disease or in relatives. Digestion, 52, 6-12.
[130a] Johnsson, C., Hallgren, R., & Tufveson, G. (1993) Recovery of hyaluronan during perfusion of small bowel transplantation reflects rejection. Transplantation, 55, 477-479.
[131a] Waldenstrom, A., Fohlman, J., Ilback, N. G., Ronquist, G., & Hallgren, R. G.-B. (1993) Coxsackie B3 myocarditis induces a decrease in energy charge and accumulation of hyaluronan in the mouse heart. Eur. J. Clin. Invest., 23, 277-282.
[132a] Wells, A., Larsson, E., Hanas, E., Laurent, T., & Hallgren, R. T.-G. (1993) Increased hyaluronan in acutely rejecting human kidney grafts. Transplantation, 55, 1346-1349.
[133a] Tufveson, G., Hallgren, R., Johnsson, C., & Wahlberg, J. Use of hyaluronidase in treatment of interstitial edema from organ grafts. Tufveson, Gunnar, Hallgren, Roger, Johnsson, Cecilia, and Wahlberg, Jan. WO 97-SE1313[WO 9808538 A1]. 1998. PCT Int. Appl., 18 pp. CODEN: PIXXD2. Jul. 24, 1997.
   Ref Type: Patent
[134a] Johnsson, C., Hallgren, R., Elvin, A., Gerdin, B., & Tufveson, G. (1999) Hyaluronidase ameliorates rejection-induced edema. TRANSPLANT INTERNATIONAL, 12, 235-243.
[135a] Johnsson, C., Hallgren, R., & Tufveson, G. (2000) Role of hyaluronan in acute pancreatitis. Surgery, 127, 650-658.
[136a] Kimata, K., Honma, Y., Okayama, M., Oguri, K., Hozumi, M., & Suzuki, S. (1983) Increased synthesis of hyaluronic acid by mouse mammary carcinoma cell variants with high metastatic potential. Cancer Res., 43, 1347-1354.
[137a] Zhang, L., Underhill, C. B., & Chen, L. (1995) Hyaluronan on the surface of tumor cells is correlated with metastatic behavior. Cancer Res., 55, 428-433.
[138a] West, D. C. & Shaw, D. M. (1998) Tumour hyaluronan in relation to angiogenesis and metastasis. In The chemistry, biology and medical applications of hyaluronan and its derivatives (Laurent, T. C., ed), pp. 227-233. Portland Press, London.
[139a] Simpson, M. A., Reiland, J., Burger, S. R., Furcht, L. T., Spicer, A. P., Oegema, T. R., Jr., & McCarthy, J. B. (2001) Hyaluronan Synthase Elevation in Metastatic Prostate Carcinoma Cells Correlates with Hyaluronan Surface Retention, a Prerequisite for Rapid Adhesion to Bone Marrow Endothelial Cells. J. Biol. Chem., 276, 17949-17957.
[140a] Knudson, W. (1996) Tumor-associated hyaluronan—Providing an extracellular matrix that facilitates invasion. Am. J. Pathol., 148, 1721-1726.
[143a] Schinkel, A. H. & Jonker, J. W. (2003) Mammalian drug efflux transporters of the ATP binding cassette (ABC) family: an overview. Adv. Drug Deliv. Rev., 55, 3-29.

1. A compound which is characterized by a formula selected from the following formulas A, B, C, D, E or F: 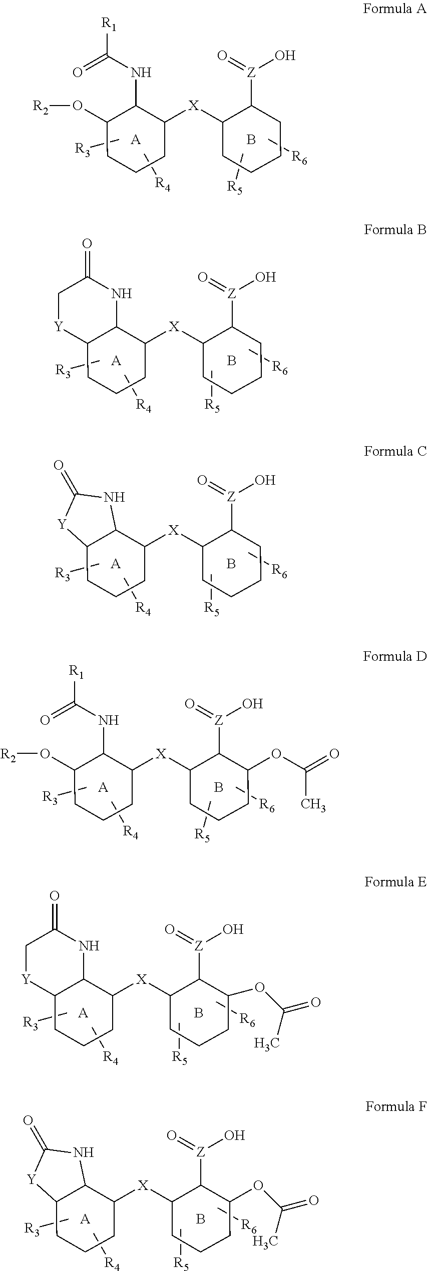
or a pharmaceutically acceptable salt thereof,
wherein
the ring systems A and B are independently selected from a monosaccharide, aryl (preferably phenyl), a heteroaryl ring or cycloalkyl (preferably cyclohexan), preferably with all substituents in equatorial configurations;
R1 is independently selected from alkyl (preferably C6-C12), a substituted or unsubstituted phenyl, preferably CH3;
R2 is H, alkyl (preferably C6 to C12), a carbohydrate in a glycosidic β-linkage, preferably H;
R3, R4, R5, and R6 are independently selected from (OH) hydroxy, alkyl (preferably C6 to C12), alkoxy (preferably C6 to C12), amino, alkylamino (preferably C6 to C12), halogen, cinnamylhydroxy, cinnamoyl, cinnamylamino, cinnamoylamino;
X is O, NH, alkylamino (NR), CO, S;
Z is C, sulfinate (S,) sulfonate (SO), phosphonate (O═P—OH), phosphinate (P—R, where R is alkyl or phenyl).

2. A pharmaceutical composition comprising one or more compound(s) selected from the following formulas A, B, C, D, E and F of claim 1 and, optionally, a pharmaceutically acceptable carrier.

3. A method for the treatment of (for treating) a disease which is associated with an excess transport of hyaluronan across a lipid bilayer comprising a step of administering one or more compound(s) selected from the following formulas A, B, C, D, E and F of claim 1 to a subject.

4. The pharmaceutical composition of claim 2 for the treatment of (for treating) a disease which is associated with an excess transport of hyaluronan across a lipid bilayer

5. The method of claim 3 wherein said disease is associated with or characterized by degeneration and/or a destruction of cartilage.

6. The method of claim 3 wherein said disease is associated with or characterized by an excess transport/export of hyaluronan of cells, selected from the group consisting of fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, epithelial cells and/or chondrocytes.

7. The method of claim 6, wherein said cell is comprised in a tissue.

8. The method of claim 7, wherein said tissue is cartilage tissue.

9. The method of claim 7, wherein said cell or said tissue is derived from a mammalian subject.

10. The method of claim 9, wherein said mammalian subject is a human, a horse, a camel, a dog, a cat, a pig, a cow, or a goat.

11. The method of claim 3 for the treatment of ischemic or inflammatory edema; tumors which are characterized by an overproduction of hyaluronan such as melanoma, mesothelioma or colon carcinoma, lump formation; injuries/conditions which are followed by inflammation and hyaluronan overproduction, alveolitis, pancreatitis, pulmonary or hepatic fibrosis, radiation induced inflammation, Crohn's disease, myocarditis, scleroderma, psoriasis, sarcoidosis and/or arthritis.

12. The method of claim 11, wherein said arthritis is osteoarthritis, (juvenile) chronic arthritis, rheumatoid arthritis, psoriatic arthritis, A. mutilans, septic arthritis, infectious arthritis and/or reactive arthritis.

13. The method of claim 3, wherein said inhibitor(s) is(are) administered prophylactically.

14. The method of claim 3, wherein said inhibitor(s) is(are) administered therapeutically.

15. A method for manufacturing a pharmaceutical composition comprising a step of formulating one or more compound(s) selected from the following formulas A, B, C, D, E and F of claim 1 in a pharmaceutically acceptable form.

16. A method of preventing, ameliorating and/or treating the symptoms of a disease which is associated with an excess transport of hyaluronan across a lipid bilayer in a subject comprising a step of administering at least one inhibitor selected from the following formulas A, B, C, D, E and F of claim 1 to a subject, preferably a mammalian subject, such that the disease which is associated with an excess transport of hyaluronan across a lipid bilayer is prevented, ameliorated and/or treated.
